(19) United States
(12) Patent Application Publication
(10) Pub. No.: US 2007/0145161 A1
(43) Pub. Date: Jun. 28, 2007

(54) Liquid discharge head and liquid discharge apparatus using the head

(76) Inventors: Manabu Tomita, Kanagawa (JP); Iwao Ushinohama, Kanagawa (JP); Shinichi Horii, Kanagawa (JP); Takeo Eguchi, Kanagawa (JP)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/548,792

(22) PCT Filed: Mar. 17, 2004
(86) PCT No.: PCT/JP04/03551

§ 371 Date: Jan. 16, 2007

(30) Foreign Application Priority Data
Mar. 20, 2003 (JP) 2003-079153

(51) Int. Cl. A01G 27/00; F23D 14/28; A61M 11/06

(52) U.S. Cl. 239/337; 239/338; 239/67

(57) ABSTRACT

The present invention relates to a liquid discharge head that controls ink discharge direction using a plurality of heating elements.

The liquid discharge head comprises, on a single semiconductor substrate (101), a plurality of heating elements (102a) (102b) that are adjacently disposed to each other in an ink liquid chamber (105) and generates bubbles in the ink supplied to the ink liquid chamber to discharge the ink from a nozzle (104a), a switching element (121a) that supplies power to the heating elements, and switching elements (121b) (121c) that control the ink discharge direction while supplying different levels of power to the heating elements or changing the timing of giving power thereto. On the semiconductor substrate, an energy supply wiring pattern (224) for supplying power to the heating elements and a control wiring pattern (236) for controlling the switching element (121a) and switching elements (121b) (121c) are provided in different conductive layers.

### TECHNICAL FIELD

The present invention relates to a liquid discharge head that discharges liquid in a liquid chamber from a discharge port using thermal energy and the like and a liquid discharge apparatus including the liquid discharge head.

This application is based upon and claims the benefit of priority from the prior Japanese Patent Application No. 2003-079153, filed on Mar. 20, 2003, the entire contents of which are incorporated herein by reference.

### BACKGROUND ART

In recent years, there has been an increasing demand for color output in the field of a hard copy, printing, and the like. To meet this demand, image formation apparatuses, liquid discharge apparatuses, and the like using a color image formation method such as dye sublimation thermal transfer, fusion thermal transfer, ink-jet, electro photography, and thermal silver-salt development have been proposed.

For example, the ink-jet type liquid discharge apparatus squirts droplets of recording liquid (ink) from a nozzle formed in a printer head which is a liquid discharge head to form dots on a recording medium, and thereby can output a high-quality image with a simple structure. In the ink-jet method, an energy generation element applies energy to ink in a liquid chamber to thereby cause an ink droplet to fly out from the nozzle. The ink-jet method is classified into an electrostatic attraction method, continuous vibration generation method (piezo method), and thermal method depending on the type of the energy generation element.

In the thermal method, a heating element is used as the energy generation element. The heating element locally applies heat (energy) to ink in the liquid chamber to generate air bubbles in the ink in the liquid chamber. The pressure caused by the bubbles pushes out the ink from the nozzle to thereby cause the ink to squirt on the recording medium. That is, in the case of the thermal method, it is possible to print out a color image with a simple structure.

The ink-jet type liquid discharge apparatus heats ink to boiling using the heating element to generate bubbles and expands the bubbles to thereby discharge liquid from an ink-discharge port. Therefore, the ink discharge direction and the like may become unstable in some cases depending on variation in the heat amount of the heating element, ink composition, ink temperature. To solve such a problem, a technique capable of controlling the ink discharge direction has been proposed in Jpn. Pat. Appln. Laid-Open Publication No. 2000-185403.

However, in the Jpn. Pat. Appln. Laid-Open Publication No. 2000-185403, there is no disclosure concerning a drive control circuit of a plurality of heating elements. In designing the drive control circuit, the following must be taken into account.

In order to discharge ink, it is necessary to instantaneously boil the ink in the liquid chamber and expand generated bubbles. Thus, it is necessary to instantaneously supply a power of about 0.5 to 1 W and, therefore, a wiring for power supply must be low resistance. To lower the resistance of the power supply wiring, it is necessary to increase the width of the wiring. In a liquid discharge head, a plurality of liquid chambers are arranged alongside, and the respective liquid chambers include a heating element for discharging ink. The liquid chambers or ink discharge ports provided in the liquid chambers are disposed very close to one another in order to print out an image with high resolution. Accordingly, the heating elements provided in the respective liquid chambers are disposed very close to one another. Therefore, in the case where the wiring for power supply to the heating elements is configured as a common wiring for supplying a plurality of heating elements with an electrical power, it is necessary to flow more current. In other words, the width of the power supply wiring needs to be increased. If one additional wiring layer is provided as the wiring for power supply to the heating elements, manufacturing efficiency may decrease.

### DISCLOSURE OF THE INVENTION

An object of the present invention is to provide a new liquid discharge head capable of solving the above problem of the conventional technique, and a liquid discharge apparatus provided with the liquid discharge head and, more particularly, to provide a liquid discharge head capable of increasing the width of a wiring for power supply to energy generation elements such as heating elements without forming an additional conductive layer, and a liquid discharge apparatus provided with the liquid discharge head.

To achieve the above object, a liquid discharge head according to the present invention includes a liquid chamber that contains liquid and a plurality of energy generation elements disposed adjacently to each other in the liquid chamber and further includes an energy generation means for generating bubbles in the liquid in the liquid chamber when each energy generation element is supplied with energy and discharging the liquid from a discharge port, a main operation control means for supplying energy to the energy generation means to generate bubbles in the liquid in the liquid chamber to thereby discharge the liquid from a discharge port, and a sub-operation control means for controlling the discharge direction of the liquid to be discharged from the discharge port while supplying different energies to the energy generation elements or changing the timing of giving energy thereto. The liquid chamber, energy generation means, main operation control means, and sub-operation control means are provided on a single semiconductor substrate. On the semiconductor substrate, an energy supply wiring that supplies power to the energy generation means and a control wiring that controls the main operation control means and sub-operation control means are provided in different conductive layers.

A liquid discharge apparatus according to the present invention includes a liquid chamber that contains liquid and a plurality of energy generation elements disposed adjacently to each other in the liquid chamber and further includes an energy generation means for generating bubbles in the liquid in the liquid chamber when each energy generation element is supplied with energy and discharging the liquid from a discharge port, a main operation control means for supplying energy to the energy generation means to generate bubbles in the liquid in the liquid chamber to thereby discharge the liquid from a discharge port, and a sub-operation control means for controlling the discharge direction of the liquid to be discharged from the discharge port while supplying different energies to the energy generation elements or changing the timing of giving energy thereto. The liquid chamber, energy generation means, main operation control means, and sub-operation control means are provided on a single semiconductor substrate. On the semiconductor substrate, an energy supply wiring that supplies power to the energy generation means and a control wiring that controls the main operation control means and sub-operation control means are provided in different conductive layers.

Additional objects and advantages of the invention will be set forth in the description which follows, and in part will be obvious from the description, or may be learned by practice of the invention.

### BRIEF DESCRIPTION OF THE DRAWINGS

FIG. 1 is a perspective view showing an ink-jet printer apparatus according to the present invention;

FIG. 2 is a perspective view showing an ink-jet print head cartridge provided in the ink-jet printer apparatus;

FIG. 3 is a cross-sectional view showing a state where an ink cartridge is attached to the ink-jet print head cartridge;

FIG. 4 is a view schematically showing a state where a supply port of an ink supply section is closed by a valve at the time when the ink cartridge is attached to the ink-jet print head cartridge;

FIG. 5 is a view schematically showing a state where a supply port of an ink supply section is opened at the time when the ink cartridge is attached to the ink-jet print head cartridge;

FIG. 6 is a plan view showing an attachment portion of the ink-jet print head cartridge;

FIG. 7 is a cross-sectional view showing a relationship between the ink-jet print head cartridge and a head chip;

FIG. 8 is a cross-sectional view showing a state where a valve of a valve mechanism in a connection portion of the ink-jet print head cartridge is closed;

FIG. 9 is a cross-sectional view showing a state where a valve of a valve mechanism in a connection portion of the ink-jet print head cartridge is opened;

FIG. 10 is a cross-sectional view showing a head chip of the ink-jet print head cartridge;

FIG. 11 is an exploded perspective view showing the head chip of the ink-jet print head cartridge;

FIG. 12 is a plan view showing the head chip of the ink-jet print head cartridge;

FIG. 13 is a plan view schematically showing a landing point of an ink droplet discharged from a head chip;

FIG. 14A is a characteristic graph showing a relationship between a difference in bubble generation time and discharge angle of an ink droplet relative to the feeding direction of a recording medium, FIG. 14B is a characteristic graph showing a relationship between the difference in bubble generation time and an dink discharge angle relative to the arrangement direction of nozzles, and FIG. 14C is a characteristic graph showing a relationship between a difference in bubble generation time and discharge angle of an ink droplet when the base current on two heating elements is set to 80 mA and deflection current is superposed on the current flowing through one of the two heating elements to deflect the ink discharge direction;

FIG. 15 is a circuit diagram for explaining a discharge direction control circuit that controls the ink discharge direction;

FIG. 16 is a plan view for explaining the circuit arrangement of the ink discharge direction control circuit which is precondition of the present invention;

FIGS. 17A and 17B are plan views each showing the circuit arrangement of the ink discharge direction control circuit according to the present invention and, more specifically, FIG. 17A is a plan view showing a state where a power supply wiring pattern is removed, and FIG. 17B is a plan view of the power supply wiring pattern;

FIG. 18 is a plan view showing an example in which a plurality of ink discharge direction control circuits are mounted in parallel on a semiconductor substrate;

FIG. 19 is a partly perspective side view showing a state where a head cap opening/closing mechanism is closed in the ink-jet printer apparatus;

FIG. 20 is a block diagram showing a control circuit of the ink-jet printer apparatus;

FIG. 21 is a characteristic graph showing density distribution given by ink droplets discharged from the head chip;

FIG. 22 is a flowchart for explaining a control method of the ink-jet printer apparatus;

FIG. 23 is a partly perspective side view showing a state where a head cap opening/closing mechanism is opened in the ink-jet printer apparatus;

FIG. 24 is a cross-sectional view showing a state where ink bubbles are generated in the head chip of the ink-jet print head cartridge; and

FIG. 25 is a cross-sectional view showing a state where an ink droplet is discharged from the nozzle by the generated ink bubbles in the head chip of the ink-jet print head cartridge.

### BEST MODE FOR CARRYING OUT THE INVENTION

An ink-jet printer apparatus to which the present invention is applied will be described below with reference to the accompanying drawings.

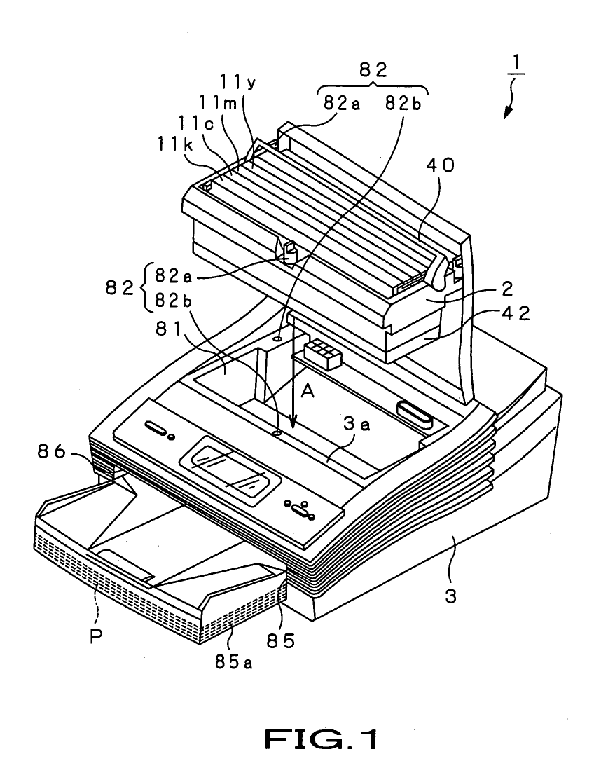

An ink-jet printer apparatus to which the present invention is applied (hereinafter, referred to as merely “printer apparatus”) 1 discharges ink or the like onto a recording paper to print an image or text, as shown in FIG. 1. The printer apparatus 1 is so-called a line type printer in which ink discharge holes are arranged according to the printing width of a recording paper P. The printer apparatus 1 includes an ink-jet print head cartridge (hereinafter referred to as merely “head cartridge”) 2 that discharges ink 4 and a printer main body 3 to which the head cartridge 2 is attached. The head cartridge 2 is detachably attached to the printer main body 3. Further, to the head cartridge 2, ink cartridges 11y, 11m, 11c, and 11k serving as ink supply sources are detachably attached. In the printer apparatus 1, a yellow ink cartridge 11y, magenta ink cartridge 11m, cyan ink cartridge 11c and black ink cartridge 11k can be used. The head cartridge 2 detachably attached to the printer main body 3 and ink cartridges 11y, 11m, 11c, and 11k detachably attached to the head cartridge are replaceable as consumable goods.

In such a printer apparatus 1, when a tray 85a that accommodates the recording papers P in a stacked manner is attached to a tray attachment port mounted on the front bottom surface side of the printer main body 3, the recording paper P in the tray 85a can be fed to the inside of the printer main body 3. When the tray 85a is attached to the tray attachment port mounted on the front surface of the printer main body 3, a paper feed/eject mechanism 84 feeds the recording paper P from a paper feed port 85 to the rear side of the printer main body 3. The feeding direction of the recording paper P that has reached the rear side of the printer main body 3 is reversed by a reverse roller and the recording paper P is fed to the front side of the printer main body 3 through the upper side of the forward passage. Texts or images are printed, according to text data or image data input from an information processing apparatus such as a personal computer, on the recording paper P that is being fed from the rear side to front side of the printer main body 3 by the time when the recording paper P is ejected from an eject port 86 mounted on the front surface of the printer main body 3.

The head cartridge 2 that prints texts or images on the recording paper P is attached to the upper surface side of the printer main body 3 in the direction denoted by the arrow A in FIG. 1 and discharges the ink 4 onto the recording paper P that is being fed by the paper feed/eject mechanism 84. Firstly, the head cartridge 2 detachably attached to the printer main body 3 that constitutes the above printer apparatus 1 and ink cartridges 11y, 11m, 11c, and 11k detachably attached to the head cartridge 2 will be described with reference to the drawings.

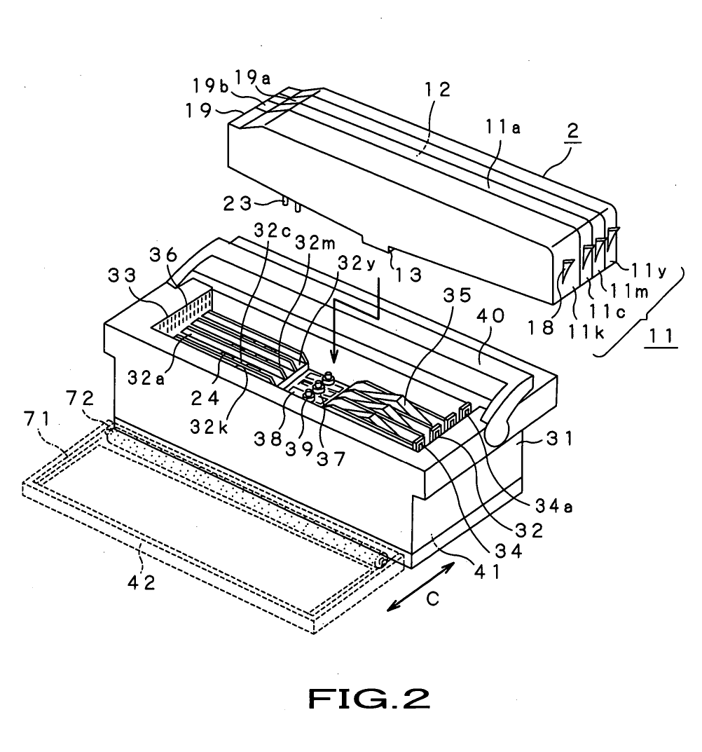

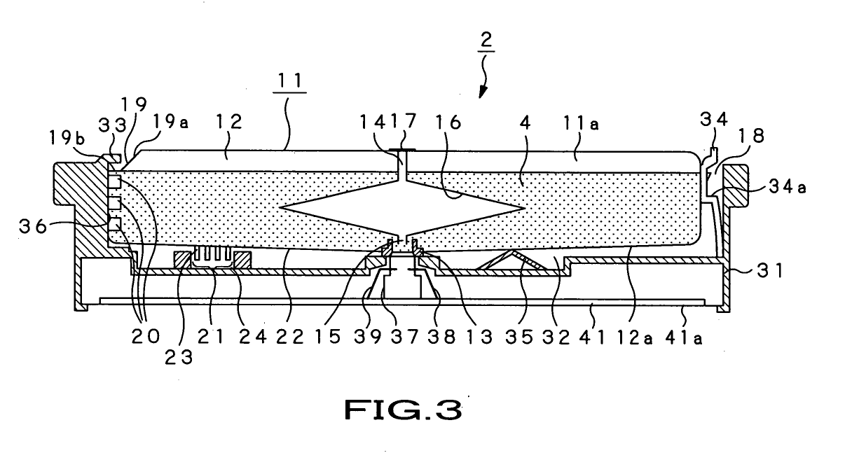

The head cartridge 2 uses, for example, an electro-thermal conversion system to discharge the ink 4 as fine droplets onto the recording media such as the recording paper P. More specifically, as shown in FIGS. 2 and 3, the head cartridge 2 includes an ink cartridge container 31, to which the ink cartridges 11y, 11m, 11c, and 11k are to be attached. The ink cartridges 11y, 11m, 11c, and 11k are vessels filled up with the ink 4 and are, hereinafter, also referred to as merely “ink cartridge 11”.

FIG. 3 shows the ink cartridge 11 detachably attached to the head cartridge 2. The ink cartridge 11 has a cartridge main body 11a formed by applying injection molding to a resin material such as polypropylene having high strength and ink resistance properties. The cartridge main body 11a is formed into substantially a rectangular shape having substantially the same width as that of the recording paper P, which maximizes the ink amount to be contained.

The cartridge main body 11a of the ink cartridge 11 includes an ink container 12 for containing the ink 4, an ink supply section 13 for supplying the ink 4 from the ink container 12 to the ink cartridge container 31 of the head cartridge 2, a communication hole 14 for taking external air into the ink container 12, an air introduction path 15 for introducing the air taken through the communication hole 14 into the ink container 12, an ink reservoir section 16 for temporarily reserving the ink 4 in the space between the communication hole 14 and air introduction path 15, a seal 17 for preventing the ink 4 from being leaked from the communication hole 14 to the outside, an engagement projection 18 and engagement step 19 by which the ink cartridge 11 is engaged with the ink cartridge container 31, a residual quantity detection section 20 for detecting the residual quantity of the ink 4 in the ink container 12, and an engagement projection portion 21 having a plurality of projections 23 for identifying the ink cartridge 11.

The ink container 12 forms the space for containing the ink 4 using a material having high air tightness. The ink container 12 is formed in substantially a rectangular shape having a dimension in longitudinal direction thereof substantially same as the width dimension of the recording medium P (dimension in the direction substantially perpendicular to the feeding direction of the recording medium P).

The ink supply section 13 is provided in substantially the center of the lower side of the ink container 12. The ink container 13 is a projecting nozzle communicating with the ink container 12. When the leading end of the nozzle is fitted to a connection portion 37 (to be described later) of the head cartridge 2, the connection between the cartridge main body 11a of the ink cartridge 11 and the ink cartridge container 31 of the head cartridge 2 is established.

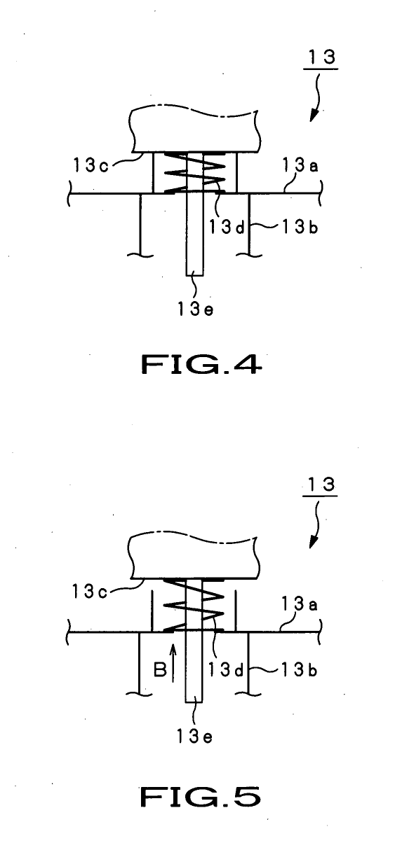

As shown in FIGS. 4 and 5, the ink supply section 13 has a supply port 13b for supplying the ink 4 on a bottom surface 13a. The ink supply section 13 further has, around the supply port 13b, a valve 13c for opening/closing the supply port 13b, a coil spring 13d for biasing the valve 13c in the closing direction of the supply port 13b, and a pin 13e for opening/closing the valve 13c. As shown in FIG. 4, before the ink cartridge 11 has been attached to the ink cartridge container 31 of the head cartridge 2, the valve 13c is biased in the closing direction of the supply port 13b for supplying the ink 4 to be connected to the connection portion 37 of the head cartridge 2 by the biasing force of the coil spring 13d serving as a biasing member to close the supply port 13b. On the other hand, as shown in FIG. 5, when the ink cartridge 11 has been attached to the ink cartridge container 31, the pin 13e is pushed up by the upper surface of the connection portion 37 of the ink cartridge container 31 that constitutes the head cartridge 2 in the direction (denoted by the arrow B in FIG. 5) opposite to the biasing direction of the coil spring 13d. As a result, the pushed up pin 13e resists the biasing force of the coil spring 13d and pushes up the valve 13c to open the supply port 13b. In this manner, the ink supply port 13 of the ink cartridge 11 is connected to the connection portion 37 of the head cartridge 2, and the ink container 12c communicates with the ink holder 51 to thereby enable the ink 4 to be supplied to the ink holder 51.

When the ink cartridge 11 is pulled out from the connection portion 37 of the head cartridge 2, that is, when the ink cartridge 11 is removed from the attachment portion 32 of the head cartridge 2, the pushed-up state of the valve 13c by the pin 13e is released, so that the valve 13c is moved in the biasing direction of the coil spring 13d to close the supply port 13b. This configuration can prevent the ink 4 in the ink container 12 from being leaked even if the leading end of the ink supply port 13 faces downward immediately before the ink cartridge 11 is attached to the ink cartridge container 31.

As shown in FIG. 3, the communication hole 14 serves as a vent hole for taking the air from the outside of the ink cartridge 11 into the ink container 12. The communication hole 14 is formed on the upper surface (in this case, in substantially the center of the upper surface) of the cartridge main body 11a, which is the position facing outside even when the ink cartridge 11 is attached to the attachment portion 32 of the head cartridge 2. With this configuration, it is possible for the ink cartridge 11 to take in the air even when being attached to the attachment portion 32 of the head cartridge 2. Through the communication hole 14, the ink cartridge 11 takes in the air by the amount corresponding to the decrease in the ink 4 in the ink container 12 from the outside into the ink cartridge 11 at the time when the ink cartridge 11 is attached to the ink cartridge container 31 to allow the ink 4 to flow down to the ink cartridge container 31 side from the ink container 12.

The air introduction path 15 connects the ink container 12 and the communication hole 14 and introduces the air taken from the communication hole 14 into the ink container 12. As a result, even when the ink 4 is supplied to the ink cartridge container 31 of the head cartridge 2 at the time when the ink cartridge 11 is attached to the ink cartridge container 31 to reduce the ink 4 in the ink container 12 to thereby reduce the pressure in the ink container 12, air is introduced through the air introduction path 15 into the ink container 12 to keep the internal pressure at equilibrium, so that it is possible to adequately supply the ink 4 to the ink cartridge container 31.

The ink reservoir section 16 is provided between the communication hole 14 and air introduction path 15 and temporarily reserves the ink 4 in order to prevent the ink 4 from flowing outside suddenly when it is leaked from the air introduction path 15 that communicates with the ink container 12.

The ink reservoir section 16 is formed into substantially a diamond shape having a longer diagonal in the longitudinal direction of the ink container 12. The air introduction path 15 is provided at the lowermost apex of the ink container 12, that is, at the portion below the shorter diagonal of the ink reservoir section 16. As a result, it is possible to return the ink 4 introduced from the ink container 12 to the ink container 12 again. The ink reservoir section 16 has the communication hole 14 at the uppermost apex of the shorter diagonal, thereby making it harder for the ink 4 introduced from the ink container 12 to be leaked from the communication hole 14.

The seal 17 is a member for sealing the communication hole 14 that prevents the ink 4 flowing back toward the communication hole 14 from being leaked outside the ink cartridge 11. Therefore, the seal 17 is made of a material having water repellency that prevents at least the ink 4 from being passed through. The seal 17 is peeled off at the time of use, and external air can be replenished through the communication hole 14 into the ink container 12 depending on the ink use amount, as needed.

The engagement projection 18 is a projection formed on the side surface of one of the narrow sides of the ink cartridge 11 and is engaged with engagement holes 34a formed on latch levers 34 of the ink cartridge container 31 of the head cartridge 2. The engagement projection 18 has an upper surface which is a plane substantially perpendicular to the side surface of the ink container 12 and a lower surface obliquely extending from the side surface to the end of the upper surface. The engagement step 19 is formed in the upper portion of the side surface opposite to the side surface on which the engagement projection 18 is formed. The engagement step 19 has an inclined plane 19a whose end has contact with one distal end of the upper surface of the cartridge main body 11a and a plane 19b extending in parallel to the upper surface of the cartridge main body 11a from the other end of the inclined surface 19a. By forming the engagement step 19, the height of the side surface on which the plane 19b is provided is made one step lower than the upper surface of the cartridge main body 11a, and thereby the ink cartridge 11 is engaged with an engagement pieces 33 of the ink cartridge container 31 by means of the step portion. When the engagement step 19 is inserted into the attachment portion 32 of the head cartridge 2, it is provided on the side surface on the insertion end side to be engaged with the engagement pieces 33 on the attachment portion 32 of the head cartridge 2. When the ink cartridge 11 is attached to the attachment portion 32 of the head cartridge 2, the engagement step 19 serves as a rotation supporting point.

As shown in FIG. 3, the residual quantity detection section 20 is provided at the side surface on which the engagement step 19 of the ink cartridge 11 is formed. The residual quantity detection section 20 includes contact members each having a pair of detection pins facing inside the ink container 12 and a contact point which is electrically connected to an ink quantity detection section 36 of the head cartridge 2 at the time when the ink cartridge 11 is attached to the attachment portion 32 of the head cartridge 2. Here, three contact members are arranged in parallel to one another in the height direction of the side surface of the cartridge main body 11a. The ink 4 has conducting properties in general, so that when a pair of detection pins facing inside the ink container 12 are dipped in the ink 4, the electrical resistivity thereof becomes low; whereas a pair of detection pins are not dipped in the ink 4, the electrical resistivity thereof becomes high. That is, when the ink container 12 is filled up with the ink 4, all the detection pins are dipped in the ink 4, with the result that all the electrical resistivity thereof become low. As the ink 4 is used, the detection pins are exposed from the ink 4 starting from the above and the electrical resistivity thereof is accordingly increased starting from the above. This configuration allows the residual quantity section 20 to detect the ink residual quantity in the ink container 12. The number of the stages of terminal plates formed in the height direction of the ink container 12 is not limited to three, but may be two. In order to detect the residual quantity more precisely, the number of the stages of the terminal plates should be increased.

The cartridge main body 11a that constitutes the ink cartridge 11 has the ink supply section 13 on the bottom surface side thereof. The bottom surface side serves as an engagement region 22 that is engaged with the attachment portion 32 of the head cartridge 2. The engagement projection portion 21 having a plurality of projections for identifying the type of the ink cartridge 11 is formed in a part of the engagement region 22 of the cartridge main body 11a. The engagement projection portion 21 can identify the type of the ink cartridge 11 based on the arrangement pattern of the plurality of projections. Only when the ink cartridges 11y, 11m, 11c, and 11k are attached to proper attachment portions 32y, 32m, 32c, and 32k of the head cartridge 2, the ink cartridges are engaged with an engagement concave portion 24 formed in the attachment portions 32y, 32m, 32c, and 32k.

The head cartridge 2 to which the above ink cartridges 11y, 11m, 11c, and 11k containing the inks 4 of yellow, magenta, cyan, and black respectively are attached will next be described.

As shown in FIGS. 2 and 3, the head cartridge 2 has the ink cartridge container 31. The ink cartridge container 31 has the attachment portions 32y, 32m, 32c, and 32k (hereinafter, referred collectively to as merely “attachment portion 32”) to which the ink cartridge 11 is attached, engagement piece 33 and latch lever 34 for fixing the ink cartridge 11, biasing member 35 for biasing the ink cartridge 11 in the removal direction, ink residual quantity detection section 36 for detecting the ink residual quantity in the ink cartridge 11, connection portion 37 which is connected to the ink supply section 13 and receives supply of the ink 4, ink detection sections 38 and 39 for detecting presence/absence of the ink 4 in the connection portion 37, a handle 40 for removing the ink cartridge container 31 from the printer main body 3, a discharge head 41 for discharging the ink 4, and a head cap 42 for protecting the discharge head 41.

The attachment portion 32 to which the ink cartridge 11 is attached is formed into substantially a concave portion with the upper surface thereof serving as an insert/eject port for the ink cartridge 11 to be attached to. In this case, four ink cartridges 11 are contained in the attachment portion side by side in the feeding direction of the recording paper P. Like the ink cartridge 11, the attachment portion 32 has a shape elongated in the printing width direction since it contains the ink cartridge 11. The ink cartridge 11 is attached to and contained in the ink cartridge container 31.

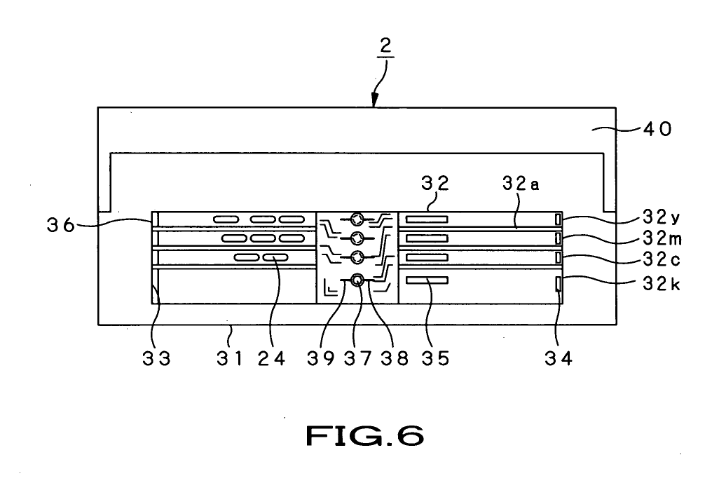

As shown in FIG. 6, the attachment portion 32 is a portion to which the ink cartridge 11 is attached. The attachment portion 32 is partitioned by partition walls 32a into the attachment portion 32y to which a yellow ink cartridge 11y is attached, the attachment portion 32m to which a magenta ink cartridge 11m is attached, the attachment portion 32c to which the cyan ink cartridge 11c is attached, and attachment portion 32k to which a black ink cartridge 11k is attached such that the attachment portions 32y, 32m, 32c and 32k are adjacently disposed to one another.

The width of the black ink cartridge 11k is made wider than the widths of other ink cartridges 11y, 11m, and 11c in order to increase the content of the ink 4. Accordingly, the width of the attachment portion 32k is made wider than the widths of the other attachment portions 32y, 32m, and 32c.

As described above and shown in FIG. 3, the engagement piece 33 is provided at the opening edges of the attachment portion 32 to which the ink cartridge 11 is attached. The engagement piece 33 is provided on the end edge of the attachment portion 32 in the longitudinal direction thereof and is engaged with the engagement step 19 of the ink cartridge 11. The ink cartridge 11 is obliquely inserted into the attachment portion 32 with the engagement step 19 side serving as an insertion end and attached to the attachment portion 32 in such a manner that one side of the ink cartridge 11 on which the engagement step 19 is not formed is rotated to the attachment portion 32 with the engagement position between the engagement step 19 and engagement pieces 33 serving as a rotation supporting point. In this manner, the ink cartridge 11 can easily be attached to the attachment portion 32. Further, it is possible to prevent the residual quantity detection section 20 from rubbing against the side surface of the ink cartridge container 31, thereby protecting the residual quantity detection section 20.

As shown in FIG. 3, the latch lever 34 is formed by bending a plate spring and is provided at the side surface opposed relative to the engagement piece 33 of the attachment portion 32, that is, at the side surface of the other end in the longitudinal direction thereof. The base end of the latch lever 34 is formed integrally with the bottom portion of the side surface of the attachment portion 32. The distal end of the latch lever 34 is elastically displaced away from/close to the side surface. The engagement hole 34 is formed on the distal end side of the latch lever 34. When the ink cartridge 11 is attached to the attachment portion 32, the latch lever 34 is elastically displaced to allow the engagement hole 34a to be engaged with the engagement projection 18 of the ink cartridge 11, thereby preventing the ink cartridge 11 attached to the attachment portion 32 from dropping out of the attachment portion 32.

The biasing member 35 is formed by bending a plate spring and is disposed in the attachment portion 32 so as to bias the ink cartridge 11 in the removal direction thereof. The biasing member 35 is an eject member having an apex portion formed by bending, the eject member being elastically displaced in the direction away from/close to the bottom surface of the ink cartridge 11 to press the bottom surface thereof, thereby biasing the ink cartridge 11 attached to the attachment portion 32 in the removal direction from the attachment portion 32. When an engagement between the engagement hole 34a and engagement projection 18 is released, the biasing member 35 ejects the ink cartridge 11 from the attachment portion 23.

The ink residual quantity detection section 36 detects the residual quantity of the ink 4 in the ink cartridge 11 in a stepwise manner. As shown in FIG. 6, four ink residual quantity detection sections 36 are provided in the attachment portions 32y, 32m, 32c, and 32k for the ink cartridges of respective colors 11y, 11m, 11c, and 11k. As shown in FIG. 3, when the ink cartridge 11 is attached to the head cartridge 2, the ink residual quantity detection section 36 comes into contact with the residual quantity detection section 20 arranged in parallel in the height direction of the side surface of the ink cartridge 11 and is electrically connected thereto. The ink residual quantity detection section 36 is pressed by a not shown biasing member in the direction toward the ink cartridge 11 side. This configuration allows the ink residual quantity detection section 36 to be attached firmly to the residual quantity detection section 20 of the ink cartridge 11 and thereby to be electrically connected thereto without fail.

The connection portions 37 are provided in substantially the center of the attachment portions 32y, 32m, 32c, and 32k. When the ink cartridges 11y, 11m, 11c, and 11k are attached to the attachment portions 32y, 32m, 32c, and 32k, the ink supply sections 13 of the ink cartridges 11y, 11m, 11c, and 11k are connected to the connection portions 37. The connection portion 37 serves as an ink supply path for supplying the ink 4 from the ink supply section 13 to the discharge head 41.

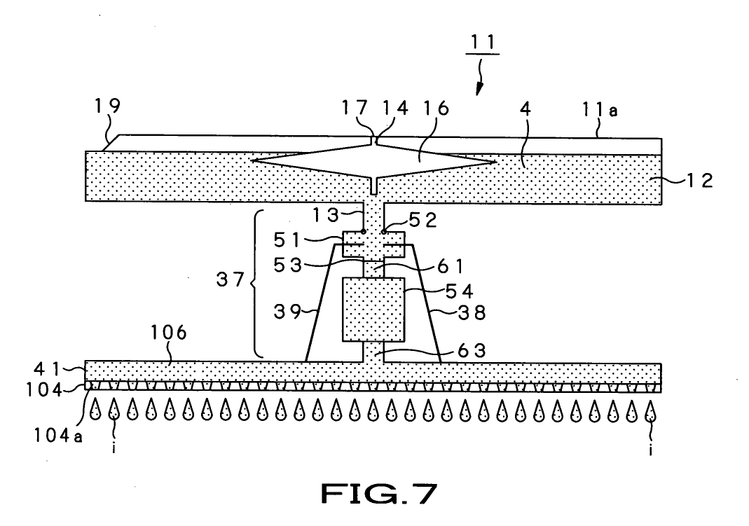

More specifically, the connection portion 37 has, as shown in FIG. 7, the ink holder 51 for holding the ink 4 supplied from the ink cartridge 11, a seal member 52 for sealing the ink supply section 13 to be connected to the connection portion 37, a filter 53 for removing impurities in the ink 4 and a valve mechanism 54 for opening/closing the supply path to the head chip 41 side.

The ink holder 51 is a space that is connected to the ink supply section 13 and holds the ink 4 supplied from the ink cartridge 11. The seal member 52 is a member provided at the upper end of the ink holder 51. When the ink supply section 13 of the ink cartridge 11 is connected to the ink holder 51 of the connection portion 37, the seal member 52 seals the portion between the ink holder 51 and ink supply section 13 so as to prevent the ink 4 from being leaked outside. The filter 53 removes dirt, dust, and the like that has been mixed into the ink 4 at the time of detachment/attachment of the ink cartridge 11. The filter 53 is provided at the portion below the ink detection sections 38 and 39.

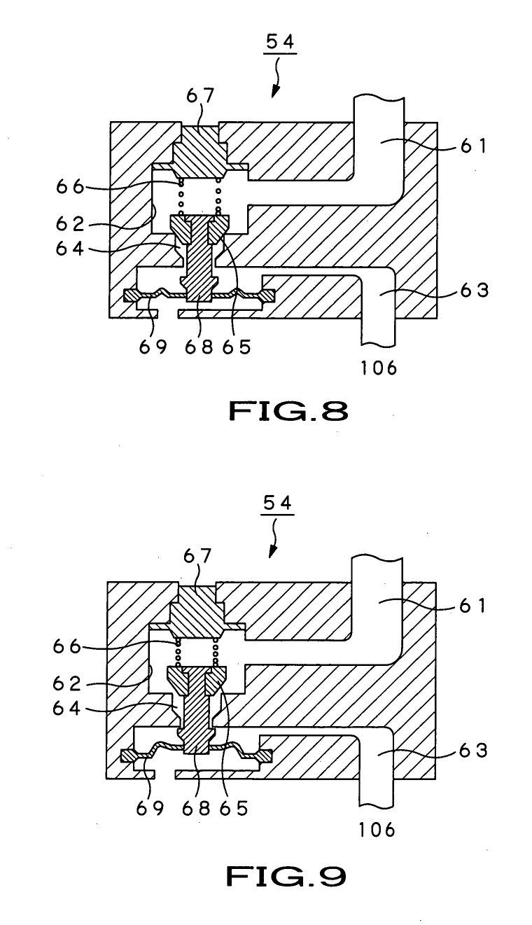

As shown in FIGS. 8 and 9, the valve mechanism 54 has an ink inflow path 61 to which the ink 4 is supplied from the ink holder 51, an ink chamber 62 into which the ink 4 flows from the ink inflow path 61, an ink outflow path 63 through which the ink 4 flows out from the ink chamber 62, an opening portion 64 provided between the ink inflow path 61 side of the ink chamber 62 and the ink outflow path side of the ink chamber 62, a valve 65 for opening/closing the opening portion 64, a biasing member 66 for biasing the valve 65 in the closing direction of the opening portion 64, a negative pressure adjusting screw 67 for adjusting the strength of the biasing member 66, a valve shaft 68 to be connected to the valve 65, and a diaphragm 69 to be connected to the valve shaft 68.

The ink inflow path 61 is a supply path connected to the ink container 12 of the ink cartridge 11 through the ink holder 51. Through the ink flow path 61, the ink 4 in the ink container 12 can be supplied to the discharge head 41. The ink inflow path 61 is formed from the bottom surface of the ink holder 51 to the ink chamber 62. The ink chamber 62 is a space having substantially a rectangular solid integrally formed with the ink inflow path 61, ink outflow path 63, and opening portion 64. The ink 4 flows into the ink chamber 62 through the ink inflow path 61, passed through the opening portion 64, and flows out of the ink chamber 62 through the ink outflow path 63. The ink outflow path 63 is a supply path to which the ink 4 is supplied from the ink chamber 62 through the opening portion 64. The ink outflow path 63 is connected to the discharge head 41. That is, the ink outflow path 63 is formed from the bottom surface of the ink chamber 62 to the discharge head 41.

The valve 65 is a valve that closes the opening portion 64 to divide the ink chamber 62 into the ink inflow path 61 side and ink outflow path 63 side. The valve 65 moves upward and downward by a biasing force of the biasing member 66, a restoring force of the diaphragm 69 connected to the valve 65 through the valve shaft 68, and a negative pressure of the ink 4 on the ink outflow path 63 side. When being at lower end, the valve 65 closes the opening portion 64 to divide the ink chamber 62 into the ink inflow path 61 side and ink outflow path 63 side, thereby blocking supply of the ink 4 to the ink outflow path 63. When being at upper end against the biasing force of the biasing member 66, the valve 65 does not divide the ink chamber 62 into the ink inflow path 61 side and ink outflow path 63 side, with the result that the ink 4 can be supplied to the discharge head 1. Although any material can be used for the valve 65, it is made of, for example, a rubber elastic body, so called an elastomer in order to ensure high sealing properties.

The biasing member 66 is, for example, a compression coil spring. The biasing member 66 is provided between the upper surface of the valve 65 and the upper surface of the ink chamber 62 and connects the negative pressure adjustment screw 67 and valve 65. The biasing force of the biasing member 66 biases the valve 65 in the closing direction of the opening portion 64. The negative pressure adjustment screw 67 is a screw for adjusting the biasing force of the biasing member 66. That is, the biasing force of the biasing member 66 can be adjusted through the adjustment of the negative pressure adjustment screw 67. As a result, although details will be described later, it is possible to adjust the negative pressure of the ink 4 for operating the valve 65 that opens/closes the opening portion 64 through the negative pressure adjustment screw 67.

The valve shaft 68 is a shaft whose one end is connected to the valve 65 and the other end is connected to the diaphragm 69. With this configuration, the valve 65 and diaphragm 69 exercise in conjunction with each other. The diaphragm 69 is a thin elastic plate connected to one end of the valve shaft 68. The diaphragm 69 is constituted by one main surface on the ink outflow path 63 side of the ink chamber 62 and the other main surface exposed to the air and elastically displaced to the air side and ink outflow path 63 side by an atmosphere pressure and a negative pressure of the ink 4.

As shown in FIG. 8, in the valve mechanism 54 having the above-described configuration, the valve 65 is pressed by a biasing force of the biasing member 66 and a biasing force of the diaphragm 69 in such a direction to close the opening portion 64 of the ink chamber 62. When the ink 4 is discharged from the discharge head 41 to increase the negative pressure of the ink 4 in the ink chamber 62 on the ink outflow path 63 side, which is one of the regions obtained by dividing the ink chamber 62 with the opening portion 64 as a border, the diaphragm 69 is pushed up by an atmosphere pressure under a negative pressure of the ink 4 as shown in FIG. 9, to thereby push up the valve shaft 68 and valve 65 against a biasing force of the biasing member 66. At this time, the opening portion 64 between the ink inflow path 61 side of the ink chamber 62 and ink outflow path 63 side is released, with the result that the ink 4 is supplied from the ink inflow path 61 side to the ink outflow path 63 side. Thereafter, the negative pressure of the ink 4 is decreased and the diaphragm 69 is restored to the original shape by its restoring force, with the result that the biasing force of the biasing member 66 moves down the valve shaft 68 and valve 65 in such a direction to close the ink chamber 62. As described above, when the negative pressure of the ink 4 is increased every time the ink 4 is discharged, the above operation is performed in the valve mechanism 54.

In the connection portion 37, the quantity of the ink 4 in the ink container 12 is reduced when the ink 4 in the ink container 12 is supplied to the ink chamber 62. At this time, however, the outside air is introduced into the ink cartridge 11 through the air introduction path 15. The air introduced into the ink cartridge 11 is sent to the upper portion of the ink cartridge 11. As a result, an ink droplet i is returned to a state before being discharged from a nozzle 104a (to be described later) to keep the internal pressure in the ink container 12 at equilibrium. This equilibrium state is obtained when there is little ink 4 in the air introduction path 15.

As shown in FIG. 7, the ink detection sections 38 and 39 are made of a pair of linear members having conducting properties, the linear members detecting the presence/absence of the ink 4 in the connection portion 37 to be connected to the ink supply section 13 of the ink cartridge 11. The leading ends of the linear members face the inside of the connection portion 37. The ink detection sections 38 and 39 are disposed in such a manner that one ends thereof penetrate the ink holder 51 from the outside of the connection portion 37 and the other ends thereof are connected to the discharge head 41.

The one ends of the ink detection sections 38 and 39 are positioned above the filter 53 in the connection portion 37. Otherwise, the negative pressure of the ink 4 on the discharge head 41 side is increased in the case where the level of the ink 4 is lower than the filter 53, causing the apparatus to malfunction. The ink detection sections 38 and 39 detect the ink 4 at the position nearer to the ink cartridge 11 than the filter 53, thereby preventing the level of the ink 4 from going below the filter 53.

The handle 40 makes it easy to remove the ink cartridge container 31 if replacement is necessary due to wear of the ink cartridge container 31 or if repair of the ink-jet printer apparatus 1 is necessary.

The discharge head 41 is disposed along the bottom surface of the ink cartridge container 31. The discharge head 41 has nozzles 104a (to be described later) linearly arranged for respective colors, the nozzles serving as ink discharge ports for discharging the ink droplet i supplied from the connection portion 37.

As shown in FIG. 2, the head cap 42 serves as a cover for protecting the discharge head 41. When the ink 4 is discharged, the head cap 42 is opened/closed by a cover opening/closing mechanism (to be described later) of the printer main body 3. The head cap 42 has a groove portion 71 formed in the opening/closing direction, and a cleaning roller 72 which is formed in the longitudinal direction of the head cap 42 and absorbs the excess ink 4 adhered to a discharge surface 41a of the discharge head 41. The head cap 42 is configured to move along the groove portion 71, that is, in the direction of the arrow C in FIG. 2 which is shorter direction of the ink cartridge 11 at the time of opening/closing operation. The cleaning roller 72 is rotated while contacting the discharge surface 41a of the discharge head 41 at the time of opening/closing operation to absorb the excess ink 4, thereby cleaning the discharge surface 41a of the discharge head 41. A member having a high water absorption rate is used for the cleaning roller 72. The head cap 42 prevents the ink 4 in the discharge head 41 from being dried.

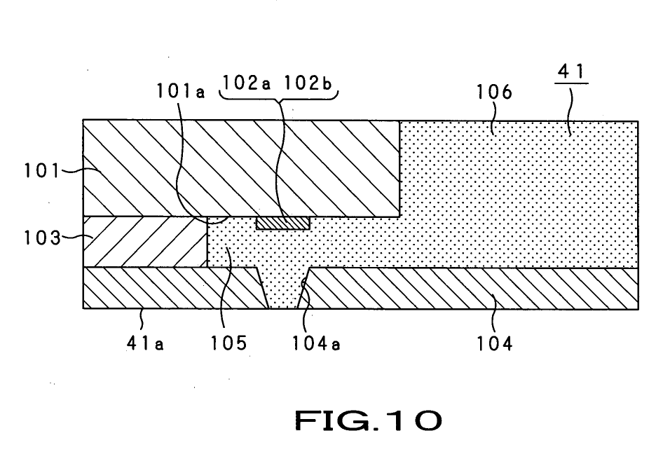

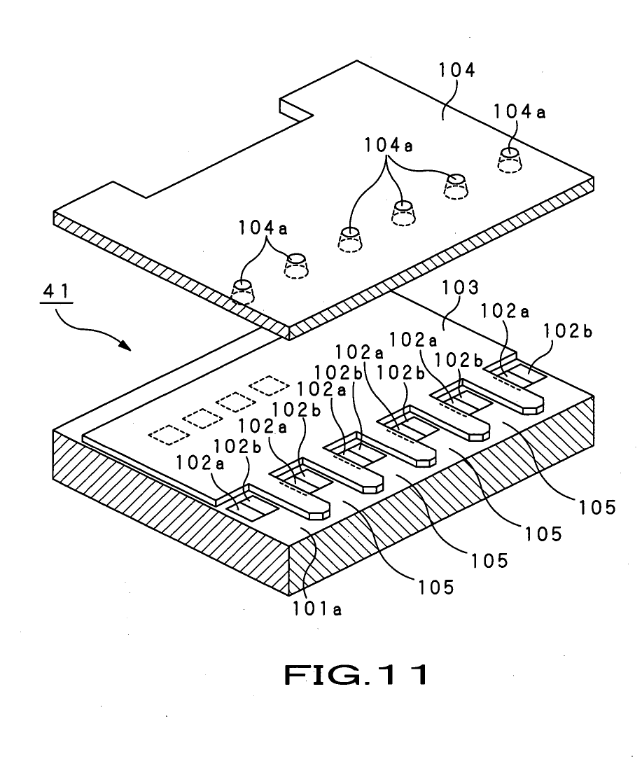

As shown in FIGS. 10 and 11, the discharge head 41 has, for ink 4 of each color, a semiconductor substrate 101 constituting a base circuit substrate, a pair of heating elements 102a and 102b for heating the ink 4, a barrier layer 103 for preventing leakage of the ink 4, a nozzle sheet 104 having a large number of nozzle 104a through which the ink 4 is discharged in a liquid droplet state, an ink liquid chamber 105 which is surrounded by the above components and receives supply of the ink 4, and an ink flow path 106 for supplying the ink 4 to the ink liquid chamber 105.

The semiconductor substrate 101 is a semiconductor substrate made of silicone and has, on one main surface 101a, the heating elements 102a and 102b thereof as well as control circuits including a main operation control circuit, sub-operation control circuit, and the like for controlling the heating elements 102a and 102b. The control circuit is constituted by a logic IC (Integrated Circuit), driver transistor, or the like.

The pair of heating elements 102a and 102b generate heat using a power supplied from the control circuit and heat the ink 4 in the ink liquid chamber 105 to increase the internal pressure in the ink liquid chamber 105. The heated ink 4 is discharged from the nozzles 104a formed on the nozzle sheet 104 (to be described later) in a liquid droplet state.

The barrier layer 103 is laminated on the main surface 101a of the semiconductor substrate 101. The barrier layer 103 is made of, for example, exposure hardening type dry film resist. After the barrier layer 103 is laminated on the entire main surface 101a of the semiconductor substrate 101, unnecessary portion is removed by a photolithography process. As a result, the barrier layer 103 surrounds respective pairs of heating elements 102a and 102b in substantially U-shape. The area in which the barrier layer 103 surrounds the pair of heating elements 102a and 102b constitute a part of the ink liquid chamber 105.

The nozzle sheet 104 is a sheet-like member on which the nozzles 104a for discharging the ink droplet i and is laminated on the side opposite to the semiconductor substrate 101 of the barrier layer 103. The nozzle 104a is a minute hole formed on the nozzle sheet 104 and opening in a circular manner. One nozzle 104a is so disposed as to face a pair of heating elements 102a and 102b. The nozzle sheet 104 constitutes a part of the ink liquid chamber 105.

The ink liquid chamber 105 is a space surrounded by the semiconductor substrate 101, pair of heating elements 102a and 102b, barrier layer 103 and nozzle sheet 104 and receives supply of the ink 4 through the ink flow path 106. The ink 4 in the ink liquid chamber 105 is heated by the heating elements 102a and 102b to increase the internal pressure in the ink liquid chamber 105. The ink flow path 106 is connected to the ink outflow path 63 of the connection portion 37 and receives supply of the ink 4 from the ink cartridge 11 connected to the connection portion 37, thereby constituting flow paths for sending the ink 4 to the respective ink liquid chambers 105 communicating with this ink flow path 106. That is, the ink flow path 106 communicates with the connection portion 37, allowing the ink 4 supplied from the ink cartridge 11 to flow into the ink flow path 106 and fill the ink liquid chamber 105.

In the abovementioned discharge head 41, a pair of heating elements 102a and 102b are provided for each ink liquid chamber 105, and about hundred ink liquid chambers 105 each having the heating element pair 102a and 102b are arranged in a line in general. The discharge head 41 appropriately selects the pair of heating elements 102a and 102b according to an instruction from a controller of the printer apparatus 1 and drives the pair, thereby discharging the ink 4 in the ink liquid chamber 105 in a liquid droplet state from the nozzle 104a corresponding to the relevant ink liquid chamber 105.

More specifically, in the discharge head 41, the ink 4 flows from the ink flow path 106 connected to the discharge head 41 into the ink liquid chamber 105. Then, a pulse current is applied for a short time, for example, 1 to 3 μsec to the pair of heating elements 102a and 102b to allow the pair to rapidly generate heat, with the result that gas phase ink bubbles are generated at the boundary between the ink 4 and the pair of heating elements 102a and 102b. Subsequently, the ink 4 is pressed by a volume corresponding to the volume of the expanded ink bubbles and, further, the ink 4 boils. As a result, the ink 4 is discharged, by a volume corresponding to the volume of the ink 4 that is pressed by the ink bubbles at the portion contacting the nozzle 104a, from the nozzle 104a as an ink droplet i, and put on the recording paper P.

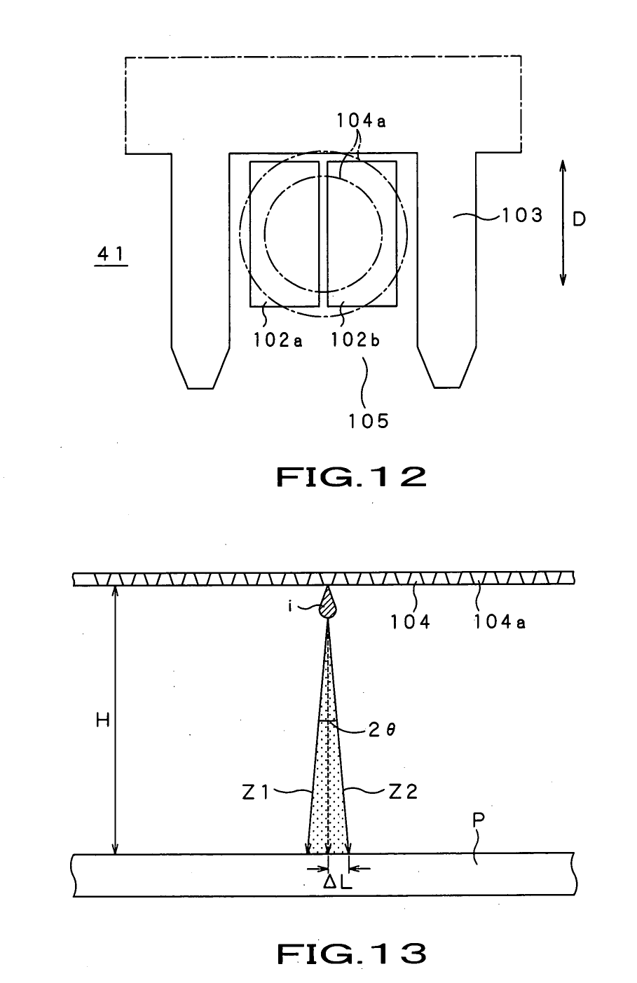

As shown in FIG. 12, in the discharge head 41, a pair of heating elements 102a and 102b are arranged side by side in one ink liquid chamber 105. That is, one ink liquid chamber 105 has a pair of heating elements 102a and 102b. More specifically, the pair of heating elements 102a and 102b are arranged side by side in the direction substantially perpendicular to the feeding direction of the recording paper P, which is denoted by the arrow D in FIG. 12. In FIG. 12, the position of the nozzle 104a is denoted by the dashed line.

The divided heating elements obtained by dividing lengthwise one heating element 102 have the same length as the original (one heating element 102) and a width half the original. Therefore, the resistance value of one of the divided heating elements 102 is double that of the original. When the divided heating elements 102 are connected in series, which means that the heating elements 102 each having a resistance value double that of the original are connected in series, the total resistance value becomes four times that of the original.

In order to boil the ink in the ink liquid chamber 105, it is necessary to apply a constant power to the heating elements 102 to heat them. The reason is that the ink is discharged using the energy at the time of boiling. When the resistance value is low, current to be applied must be increased. In this case, however, the resistance value of the heating elements 102 is made higher, so that it is possible to boil the ink with reduced current.

With the above configuration, it is possible to reduce the size of a transistor for supplying current, resulting in space reduction. When the thickness of the heating elements 102 is made smaller, the resistance value thereof can be increased. However, in the light of the material selected as the heating elements 102, strength, and endurance, there is a limit to reduce the thickness of the heating elements 102. Therefore, in the present invention, not by reducing the thickness but by dividing one heating element 102 into two, the resistance value is made higher.

When the divided heating elements 102 provided in one ink liquid chamber 105 are allowed to simultaneously reach the temperature at which the ink boils, that is, the time needed for bubble generation is made equal between the heating elements 102, the inks on the two heating elements 102 boils simultaneously and, therefore, the ink droplet is discharged in the center axis direction of the nozzle 104a.

On the other hand, when time difference is given to the bubble generation time of the divided heating elements 102, the inks on the two heating elements 102 do not boil simultaneously. As a result, the discharge direction of the ink droplet is deviated from the center axis direction of the nozzle 104a and the ink droplet is discharged in a deflecting manner. Thus, it is possible to put the ink droplet on the position deviated from the normal ink landing position obtained in the case where the ink discharge direction is not deflected.

FIG. 13 is a view for explaining the deflection of the discharge direction of the ink droplet. In FIG. 13, when the ink droplet i is discharged perpendicular to the discharge surface of the ink droplet i, the ink droplet i is put on the recording medium P without deflection as indicated by the dotted arrow. On the other hand, assume that the discharge direction of the ink droplet i is deflected to deviate the discharge angle from the perpendicular position by θ (Z1 or Z2 direction in FIG. 13). In this case, the landing position of the ink droplet i is deviated by
ΔL=H×tan θ
where H (nearly constant) is a distance between the discharge surface and the surface (landing surface of the ink droplet i) of the printing paper P which is a recording medium.

As described above, when the discharge direction of the ink droplet i is deviated from the perpendicular direction by θ, the landing position of the ink droplet i is deviated by ΔL.

The distance H between the end of the nozzle 104a and printing paper P is about 1 to 2 mm, in the case of a normal ink-jet printer. Therefore, it is assumed that the distance H is fixedly set to 2 mm.

The reason for setting the distance H to substantially the fixed value is that a change in the distance H changes the landing position of the ink droplet i. That is, when the ink droplet i is discharged perpendicular to the printing paper P from the nozzle 104a, the landing position of the ink droplet is not changed even if the distance H is changed to a certain degree. On the other hand, the discharge direction of the ink droplet i is deflected as described above, the landing position of the ink droplet i is changed due to the change in the distance H.

When the resolution of the discharge head 41 is set to 600 DPI, the interval between the adjacently disposed nozzles 104a is
25.40×1000/600≈42.3 (μm).

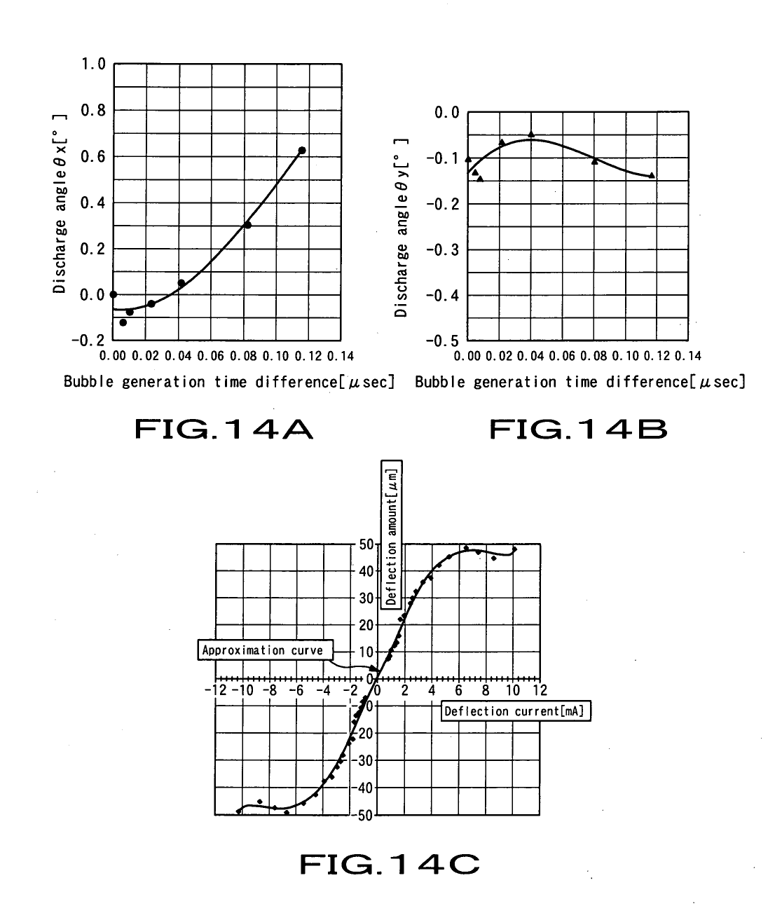

FIGS. 14A and 14B are graphs each showing a relationship between a difference in bubble generation time between the divided heating elements 102a and 102b and ink discharge angle. These graphs are obtained with the help of a simulation on a computer. In these graphs, X-direction (graph's vertical axis Ox) (note; X-direction does not mean graph's horizontal axis) denotes arrangement direction (arrangement direction of heating elements 13) of the nozzles 104a, and Y-direction (graph's vertical axis θy) (note; Y-direction does not mean graph's vertical axis) denotes the direction (feeding direction of printing paper) perpendicular to the X-direction. FIG. 14C is actual measurement data showing a relationship between a difference in bubble generation time between the divided heating elements 102a and 102b and ink discharge angle (X-direction). More specifically, deflection current obtained by dividing the difference in the current between the divided heating elements 102a and 102b by two is set as the difference in bubble generation time and plotted on the horizontal axis, and deflection amount (H is set to about 2 mm) on the ink landing position is plotted as the ink discharge angle (X-direction) on the vertical axis. In FIG. 14C, the base current on two heating elements 102a and 102b is set to 80 mA and deflection current is superposed on the current flowing through one of the two heating elements to deflect the ink discharge direction.

In the case where there is a difference in the bubble generation time between the heating elements 102 obtained by dividing one heating element 102 in the arrangement direction of the nozzle 104a, the ink discharge angle does not become perpendicular to the ink landing surface as shown in FIG. 14A. Further, as can be seen from FIG. 14A, the ink discharge angle θx (deviation from the perpendicular, which corresponds to 0 in FIG. 13) relative to the arrangement direction of the nozzle 104a is increased with an increase in the bubble generation time difference.

As described above, when the heating element 102 is divided in two, and the current supplied to the respective heating elements 102 is made different from each other, it is possible to cause a difference in bubble generation time between the two heating elements 102. Further, it is possible to deflect the ink discharge direction depending on the time difference.

As described above, the discharge head 41 can deflect the ink discharge direction. As a result, even if, for example, the resistance value varies due to manufacturing error of the heating elements 102a and 102b and the discharge direction of the ink droplets varies to make the ink landing point inaccurate, it is possible to compensate this.

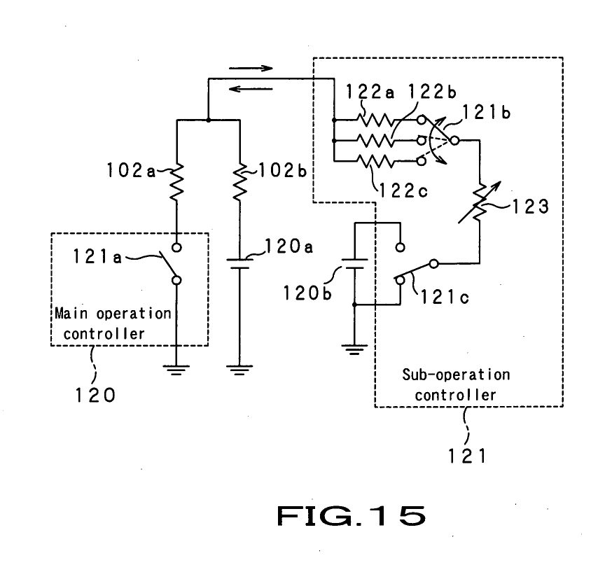

On the semiconductor substrate 101 constituting the discharge head 41, a discharge control circuit for controlling the discharge of the ink in the ink liquid chamber 105 is mounted. As shown in FIG. 15, the discharge control circuit includes power sources 120a and 120b, for supplying current to the pair of heating elements 102a and 102b each of which constitutes a resistive body, a switching elements 121a, 121b, and 121c for turning ON/OFF an electrical connection between the pair of heating elements 102a, 102b and power sources 120a, 120b, resistors 122a, 122b, and 122c for controlling the current to be supplied to the pair of heating elements 102a, 102b, and a variable resistor 123.

The power source 120a is connected to the heating element 102b. The power source 120b is selectively connected to the resistors 122a, 122b, and 122c through the switching element 121c, variable resistor 123.

The switching element 121a, which is constituted by a transistor, is disposed between the heating element 102a and the ground and functions as a main operation controller 120 for controlling ON/OFF of the heating elements 102a and 102b. The switching element 121b, which is constituted by a transistor, is connected between the variable resistor 123 and resistors 122a, 122b, and 122c and controls the current to be supplied to the heating element 102a. The switching element 121c is connected between the variable resistor 123 and power source 120b and controls the discharge direction of the ink droplet i. The resistors 122a, 122b, and 122c, variable resistor 123, switching element 121b, and switching element 121c function as a sub-operation controller 121 for controlling the discharge direction of the ink droplet i.

The resistors 122a, 122b, and 122c have resistance values different from one another and control the current to be supplied to the heating element 102a depending on the switching state of the switching element 121b. The resistance value increases in the order of resistor 122a, resistor 122b, and resistor 122c. The current to be supplied to the heating element 102a is determined depending on the resistor (122a to 122c) that the heating element 102a is connected to.

The variable resistor 123 is connected to one of the resistors 122a, 122b, and 122c to further adjust the current to be supplied to the heating element 102a.

When the switching element 121a is turned ON under the condition that the switching element 121b is turned OFF to disconnect the resistors 122a, 122b, 122c and the pair of heating elements 102a and 102b, current is supplied from the power source 120a to the serially connected pair of heating elements 102a and 102b. At this time, current does not flow in the resistors 122a, 122b, and 122c. Since the pair of heating elements 102a and 102b have the same resistance value, they generate the same heating value. Therefore, the bubble generation time is the same between the pair of heating elements 102a and 102b, with the result that the ink droplet i is discharged from the nozzle 104a such that the discharge angle of the ink 4 becomes perpendicular to the recording paper P as indicated by the dotted arrow in FIG. 13.

When the switching element 121b is connected to one of the resistors 122a, 122b, and 122c, the switching element 121a is turned ON, and the switching element 121c is connected to the ground, it is possible to change the discharge direction of the ink droplet i to the direction indicated by the arrow Z1 or Z2 in FIG. 13. That is, when the switching element 121b is connected to one of the resistors 122a, 122b, and 122c, the current to be supplied to the heating element 102a is reduced to make a difference in the current to be supplied between the pair of heating elements 102a and 102b, resulting in difference in the heating value that they generate. Here, the resistors 122a, 122b, and 122c have resistance values different from one another, so that it is possible to change the current to be supplied to the heating element 102a at three levels by the switching of the switching element 121b. As a result, the discharge head 41 can make a difference in the heating value that the pair of heating element 102a and 102b generate and make a three-level difference in the bubble generation time between the pair of heating element 102a and 102b by the switching of the switching element 121b, thereby changing the discharge direction of the ink droplet i at three levels in the arrangement direction of the pair of heating element 102a and 102b.

Further, by making the resistance value variable using the variable resistor 123, it is possible to finely control the current to be supplied to the heating element 102a. Accordingly, the discharge direction of the ink droplet i can be adjusted to control the ink landing point.

When the switching element 121c is switched to connect to the power source 120b, the discharge direction of the ink droplet i can be reversed. In this case, the current from the power sources 120a and 120b is supplied to the heating element 102a. This is the reverse of the case where the switching element 121c is connected to the ground. As a result, the ink droplet i is discharged onto the landing position on the opposite side with respect to the landing position perpendicular to the nozzle 104a with the discharge direction changed at three levels.

As described above, in the discharge control circuit, the switching of the switching elements 121b and 121c constituting the sub-operation controller 121 allows the discharge direction of the ink droplet i from the nozzle 104a to be changed at seven levels in the direction perpendicular to the feeding direction of the recording paper P. Further, the combination of the resistors 122a, 122b, 122c and variable resistor 123 allows the discharge direction of the ink droplet i to be changed at seven levels or more.

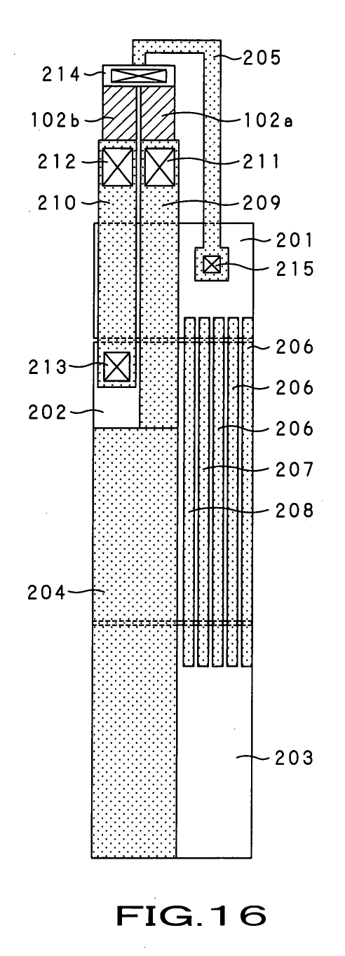

Next, a description will be made of the circuit arrangement of the above discharge control circuit mounted on the semiconductor substrate 101. The circuit arrangement is shown in FIG. 16. More specifically, the pair of heating elements 102a and 102b are disposed on one end of the semiconductor substrate 101; disposed adjacently to the heating elements 102a and 102b is a sub-operation control element formation area 201 in which the sub-operation controller including the resistors 122a, 122b, 122c, variable resistor 123, switching element 121b, switching element 121c and controlling the discharge direction of the ink droplet i is formed; disposed adjacently to the sub-operation control element formation area 201 is a main operation control element formation area 202 in which the main operation controller controlling ON/OFF of the heating elements 102a and 102b is formed; and disposed adjacently to the main operation control element formation area 202 is a control circuit element formation area 203 in which a control circuit and the like controlling the switching elements 121b and 121c that constitute the sub-operation controller are formed.

In the case of the circuit arrangement shown in FIG. 16, circuit elements such as the switching element 121a (main operation control element formation area 202) constituted by a transistor, switching elements 121b and 121c, resistors 122a, 122b, 122c (sub-operation control element formation area 201) each constituted by a transistor, a transistor, capacitor, resistor constituting the control circuit element formation area 203 are formed on the silicone substrate of the semiconductor substrate 101. Further, a power supply wiring pattern 204 for supplying power to the heating elements 102a and 102b through a not-shown insulating film is formed.

The power supply wiring pattern 204 is the uppermost conductive layer. In addition to the power supply wiring pattern 204, the following wiring patterns are formed as the uppermost conductive layer: a connection pattern 205 that connects the middle point between the pair of heating elements 102a, 102b and resistors 122a, 122b, 122c disposed on the sub-operation control element formation area 201; three control wiring patterns 206, 206, 206 that connect the control circuit element formation area 203 in which the control circuit and the like are formed and sub-operation control element formation area 201 and control the switching element 121b formed in the sub-operation control element formation area 201; a plus power wiring pattern 207 and minus power wiring pattern 208 for driving the elements 121a, 121b, 121c, 122a, 122b, and 122c; a first wiring pattern 209 that connects the power supply wiring pattern 204 and heating element 102a; and a second wiring pattern 210 that connects the heating element 102b and switching element 121a of the main operation control element formation area 202. In FIG. 16, the uppermost wiring patterns are indicated by a dot pattern.

The power supply wiring pattern 204 and the first wiring pattern 209 are continuously formed. The first wiring pattern 209 is connected to the heating element 102a through an electrode 211. One end of the second wiring pattern 210 is connected to the heating element 102b through the electrode 212 and the other end thereof is connected to a conductive layer connected to the switching element 121a of the main operation control element formation area 202 through a contact hole 213. The heating elements 102a and 102b are connected in series through an electrode 214, the electrode 214 being connected to one end of the connection pattern 205. The other end of the connection pattern 205 is connected, through a contact hole 215, to a conductive layer connected to the resistors 122a, 122b, 122c of the sub-operation control element formation area 201 of the lower layer. One ends of the control wiring patterns 206, 206, 206, plus power wiring pattern 207 and minus power wiring pattern 208 are connected, through a contact hole, to the sub-operation control element formation area 201 of the lower layer and other ends thereof are connected, through a contact hole, to the control circuit element formation area 203 of the lower layer.

In the circuit arrangement shown in FIG. 16, the heating elements 102a, 102b and the sub-operation control element formation area 201 can adjacently be disposed. On the other hand, in this circuit arrangement, the main operation control element formation area 202 is disposed between the sub-operation control element formation area 201 and control circuit element formation area 203, so that it is necessary to form the control wiring patterns 206, 206, 206, in such a manner to extend across the main operation control element formation area 202, which makes it impossible to widely form the power wiring pattern 204 to be formed in the same layer as the control wiring patterns 206, 206, 206. The power wiring pattern 204 for power supply needs to supply power of about 0.5 to 1 W to the heating elements 102a, 102b. If the width of the power wiring pattern 204 is small, the power wiring pattern 204 generates heat to adversely affect the peripheral area.

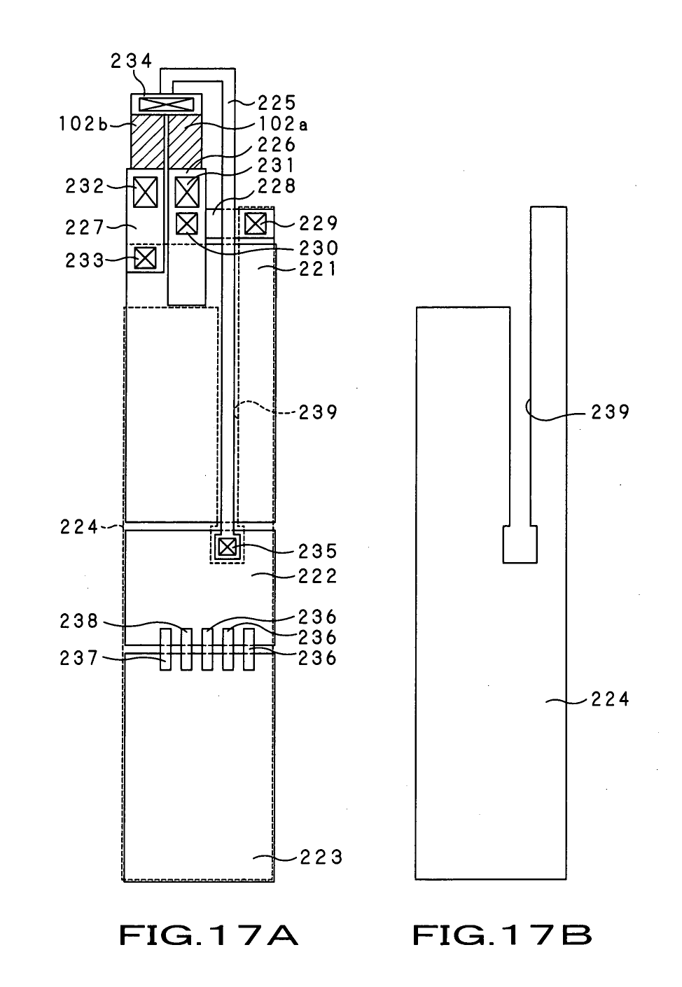

To cope with the above problem, a discharge direction control circuit is formed on the semiconductor substrate 101, as shown in FIG. 17A. In this circuit arrangement, the pair of heating elements 102a and 102b are disposed on one end of the semiconductor substrate 101; disposed adjacently to the heating elements 102a and 102b is a main operation control element formation area 221 in which the main operation controller controlling ON/OFF of the heating elements 102a and 102b is formed; disposed adjacently to the main operation control element formation area 221 is a sub-operation control element formation area 222 in which the sub-operation controller including the resistors 122a, 122b, 122c, variable resistor 123, switching element 121b, switching element 121c and controlling the discharge direction of the ink droplet i is formed; and disposed adjacently to the sub-operation control element formation area 201 is a control circuit element formation area 223 in which a control circuit and the like controlling the switching elements 121b and 121c that constitute the sub-operation controller are formed.

That is, the switching element 121a constituted by a transistor is formed in the main operation control element formation area 221 on the silicone substrate of the semiconductor substrate 101, the switching elements 121b and 121c and resistors 122a, 122b, 122c each of which is constituted by a transistor are formed in the sub-operation control element formation area 222, and the circuit elements such as the transistor, capacitor, resistor constituting the control circuit are formed on the control circuit element formation area 223. Further, on the semiconductor substrate 101 on which the above circuit elements are mounted, a lower conductive layer for connecting to the uppermost conductive layer through an insulating layer is formed. Further, an upper conductive layer is formed on the lower conductive layer with an insulating layer interposed therebetween. As the upper layer conductive layer formed on the second insulating layer, a power supply wiring pattern 224 is formed over the entire surface. Further, in the upper conductive layer, a connection pattern 225 that connects the middle point between the pair of heating elements 102a, 102b and resistors 122a, 122b, 122c disposed on the sub-operation control element formation area 222, a first wiring pattern 226 that connects the power supply wiring pattern 224 and heating element 102a, and a second wiring pattern 227 that connects the heating element 102b and switching element 121a of the main operation control element formation area 221 are formed.

The power supply wiring pattern 224 and first wiring pattern 226 that supplies current to the heating element 102a are connected to each other through the connection pattern 228 which is a lower conductive layer. That is, the power supply wiring pattern 224 which is an upper conductive layer is connected to the connection pattern 228 of the lower conductive layer through a contact hole 229 formed in an insulating layer between the upper and lower conductive layer. Further, the first wiring pattern 226 of the upper conductive layer is connected to the connection pattern 228 of the lower conductive layer through the contact hole 229 formed in an insulating layer between the upper and lower conductive layers. The first wiring pattern 226 is connected to the heating element 102a through an electrode 231. One end of the second wiring pattern 227 is connected to the heating element 102b through an electrode 232, and the other end of thereof is connected to the lower conductive layer connected to the switching element 121a of the main operation control element formation area 221 through a contact hole 233. The heating elements 102a and 102b are connected in series through an electrode 234, the electrode 234 being connected to one end of the connection pattern 225 of the upper conductive layer. The other end of the connection pattern 225 that is the upper conductive layer is connected, through a contact hole 235, to the lower conductive layer connected to the resistors 122a, 122b, 122c of the sub-operation control element formation area 222. In order to widely form the power supply wiring pattern 224, a cut portion 239 is formed in the region for the connection pattern 225, as shown in FIG. 17B.

In addition to the connection pattern 228 that connects the power supply wiring pattern 224 and first wiring pattern 226, three control wiring patterns 236, 236, 236 that connect the control circuit element formation area 223 in which the control circuit and the like are formed and sub-operation control element formation area 222 and control the switching element 121b formed in the sub-operation control element formation area 222, and a plus power wiring pattern 237 and minus power wiring pattern 238 for driving the elements 121a, 121b, 121c, 122a, 122b, and 122c are formed as the lower conductive layer. One end of the control wiring patterns 236, 236, 236, plus power wiring pattern 237, and minus power wiring pattern 238 are connected to the sub-operation control element formation area 222, and the other ends thereof are connected to the control circuit element formation area 223.

In the circuit arrangement shown in FIG. 17A, the control wiring patterns 236, 236, 236, plus power wiring pattern 237, and minus power wiring pattern 238, which are formed in the upper conductive layer in FIG. 16, are formed in the lower conductive layer, so that it is possible to widely form the power supply wiring pattern 224 in the upper conductive layer. Since the widely formed power supply wiring pattern 224 has a low resistance value, it is possible to suppress the heat generation, thereby minimizing the adverse affect on the peripheral region at which other elements and the like are formed. In particular, in the circuit arrangement shown in FIG. 17A, the main operation control element formation area 221, sub-operation control element formation area 222, and control circuit element formation area 223 are disposed in the order mentioned starting from the heating elements 102a and 102b side, so that it is possible to make the connection pattern 225 that connects the middle point between the pair of heating elements 102a, 102b and the sub-operation control element formation area 222 longer than the connection pattern 205 shown in FIG. 16, in which the sub-operation control element formation area 222 is disposed adjacently to the heating elements 102a and 102b. That is, in the circuit arrangement shown in FIG. 16, the heat generated by the heating elements 102a and 102b is transferred, through the connection pattern 205 and contact hole 215, to the sub-operation control element formation area 201, so that the sub-operation control element formation area 201 may be damaged by the heat in some cases; whereas in the circuit arrangement shown in FIG. 17, the connection pattern 225 of the upper layer is made longer, the heat from the heating elements 102a and 102b can sufficiently be radiated, thereby protecting the sub-operation control element formation area 222 from the heat.

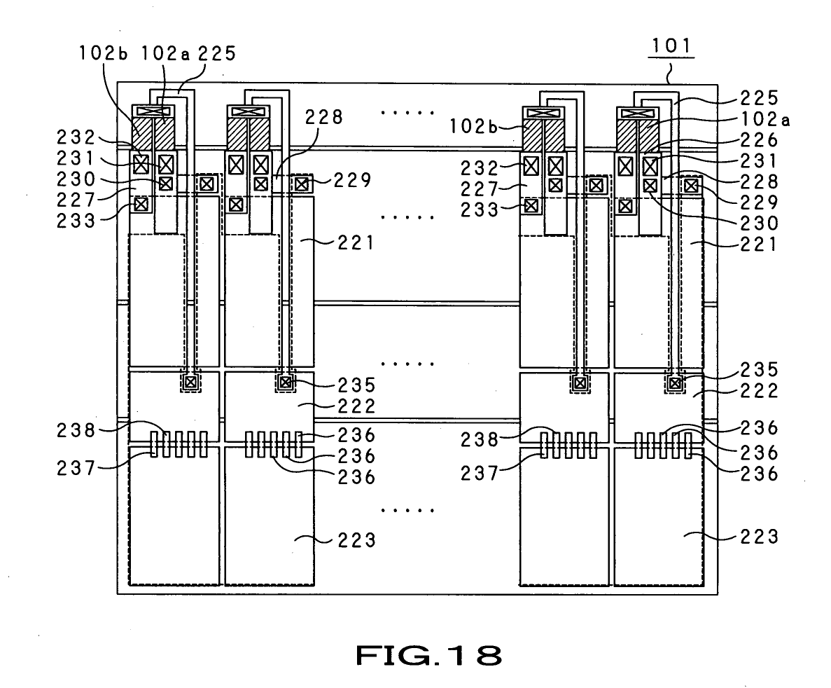

The circuit arrangement shown in FIG. 17A is designed to correspond to the pair of heating elements 102a and 102b. Here, in this circuit arrangement of FIG. 17A, the power supply wiring pattern 224 can widely be formed, so that a plurality of pairs of heating elements 102a and 102b may be arranged on one semiconductor substrate 101 as shown in FIG. 18. In this case, the power supply wiring patterns 224 that supply power to the respective pairs of heating elements 102a and 102b can be configured as a common wiring pattern. That is, power is supplied from one power supply wiring pattern 224 to a plurality of discharge direction control circuit, thereby simplifying the wiring pattern.

The printer main body 3 of the printer apparatus 1, to which the head cartridge 2 having the above configuration is attached, will next be described with reference to the drawings.

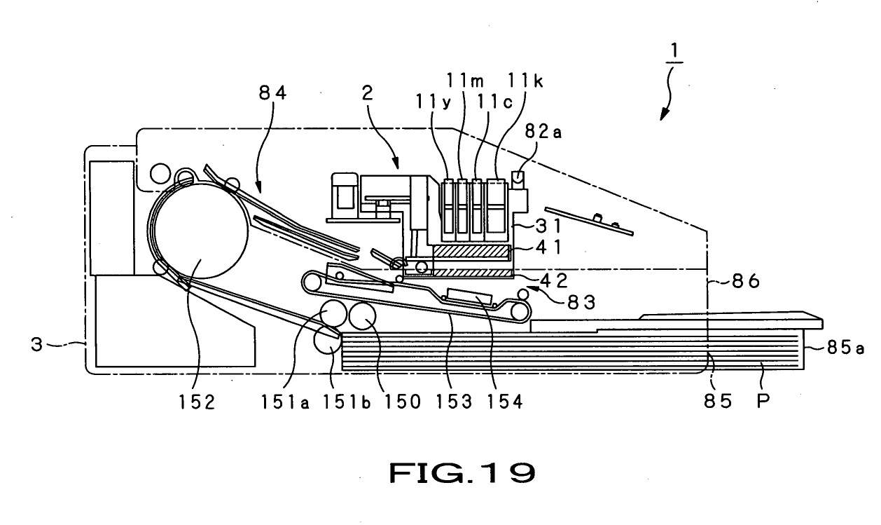

As shown in FIG. 1 and FIG. 19, the printer main body 3 includes a head cartridge attachment portion 81 to which the head cartridge 2 is attached, a head cartridge holder mechanism 82 for holding the head cartridge 2 and fixing it to the head cartridge attachment portion 81, a head cap opening/closing mechanism 83 for opening/closing a head cap 42, a paper feed/eject mechanism 84 for feeding and ejecting the recording paper P, a paper feed port 85 for supplying the recording paper P to the paper feed/eject mechanism 84, and a paper eject port 86 to which the recording paper P is output from the paper feed/eject mechanism 84.

The head cartridge attachment portion 81 is a concave portion to which the head cartridge 2 is attached. The head cartridge 2 is attached to the head cartridge attachment portion 81 such that the discharge surface 41a of the discharge head 41 is set parallel to the surface of the fed recording paper P in order to obtain a proper printing result according to data. There may be a need to replace the head cartridge 2 with a new one in some cases due to ink blockage caused in the discharge head 41. Thus, although the replacement frequency is less than the ink cartridge 11, the head cartridge 2 is a consumable goods, so that it is held by the head cartridge holder mechanism 82 in a detachable manner to the head cartridge attachment portion 81. The head cartridge holder mechanism 82 is a mechanism for holding the head cartridge 2 in a detachable manner to the head cartridge attachment portion 81. When a tab 82a on the head cartridge 2 is engaged with a not shown biasing member such as a spring provided in an engagement hole 82b of the printer main body 3, the head cartridge 2 is press-fit to a reference surface 3a on the printer main body 3. As a result, the head cartridge 2 can be positioned and fixed.

The head cap opening/closing mechanism 83 has a drive section for opening/closing the head cap 42 of the head cartridge 2. When printing is performed, the drive section moves the head cap 42 to allow the discharge head 41 to face the recording paper P. When printing is ended, the drive section closes the head cap 42 for protecting the discharge head 41. The paper feed/eject mechanism 84 has a drive section for feeding the recording paper P. The drive section feeds the recording paper P supplied from the paper feed port 85 to the discharge head 41 of the head cartridge 2, further feeds the recording paper P onto which the ink 4 has been discharged to the paper eject port 86, and outputs the recording paper P to the outside of the apparatus. The paper feed port 85 is an opening for supplying the recording paper P to the paper feed/eject mechanism 84, and a plurality of recording papers P can be stacked in the tray 85a or the like. To the paper eject port 86, the recording paper P onto which the ink droplets i have been discharged is fed by the paper feed/eject mechanism 84 and ejected to the outside of the apparatus.

A control circuit that controls the printing operation performed by the printer apparatus 1 having the above configuration will be described with reference to the drawings.

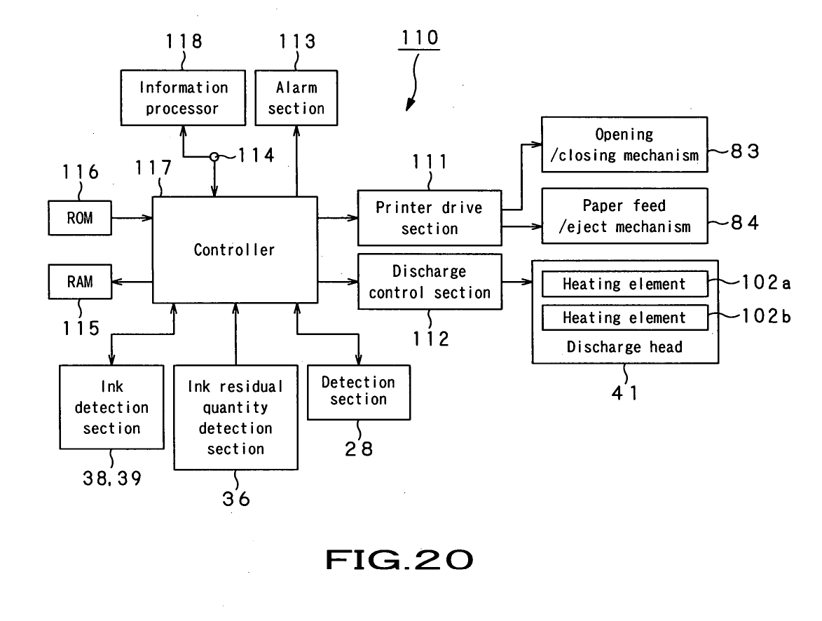

As shown in FIG. 20, a control circuit 110 includes a printer drive section 111 that drives drive sections in the printer main body 3, a discharge control section 112 that controls current and the like to be supplied to the discharge heads 41 corresponding to inks 4 of respective colors, an alarm section 113 that indicates the residual quantity of the inks 4 of respective colors, an input/output terminal 114 through which the printer apparatus 1 exchanges signals with an external device, an ROM (Read Only Memory) 116 that stores a control program and the like, an RAM (Random Access Memory) 115 that reads in the readout control program and the like, and a controller 117 that controls the above components.

The printer drive section 111 drives a drive motor that constitutes the head cap opening/closing mechanism 83 in response to a control signal from the controller 117 to open/close the head cap 42. Further, the printer drive section 111 drives a drive motor that constitutes the paper feed/eject mechanism 84 in response to a control signal from the controller 117 to feed the recording paper P from the paper feed port 85 and eject the recording paper P from the paper eject port 86 after printing.

The discharge control section 112 is constituted by the discharge direction control circuit which has been described with reference to FIG. 15. The alarm section 113 is a display means such as an LCD (Liquid Crystal Display). The alarm section 113 displays information such as printing condition, printing state, ink residual quantity. The alarm section 113 can be a voice output means such as a speaker. In this case, the information such as printing condition, printing state, ink residual quantity is output by voice. Further, it is possible to combine the display means and voice output means. The alarm may be made through a monitor or speaker of an information processor 118.

The input/output terminal 114 transmits the information such as printing condition, printing state, ink residual quantity to the external information processor 118 and the like through an interface. Further, the input/output terminal 114 receives an input of a control signal for outputting the information such as printing condition, printing state, ink residual quantity from the external information processor 118 and the like or printing data. The abovementioned information processor 118 is, for example, an electronic apparatus such as a personal computer or PDA (Personal Digital Assistant).

A serial interface, parallel interface, or the like can be used as the interface of the input/output terminal 114 which is connected to the information processor 118 or the like. More specifically, interfaces conforming to USB (Universal Serial Bus), RS (Recommended Standard) 232C, IEEE (Institute of Electrical and Electronic Engineers) 1394 can be used. The data communication between the input/output terminal 114 and information processor 118 can be performed through a wired or wireless communication. Examples of the wireless communication standard include IEEE 802.11a, 802.11b, 802.11g.

The ROM 116 is a memory such as an EPROM (Erasable Programmable Read-Only Memory) and stores a program of processes that the controller 117 performs. The stored program is loaded by the controller 117 into the RAM 116. The RAM 115 stores the program that the controller 117 reads out from the ROM 116 or state of the printer apparatus 1.

A network such as the Internet may be interposed between the input/output terminal 114 and information processor 118. In this case, the input/output terminal 114 is connected to a network such as LAN (Local Area Network), ISDN (Integrated Services Digital Network), xDSL (Digital Subscriber Line), FTHP (Fiber To The Home), CATV (Community Antenna Television), or BS (Broadcasting Satellite) and data communication is performed according to various protocols such as TCP/IP (Transmission Control Protocol/Internet Protocol).

The controller 117 controls the components based on printing data and a control signal input from the input/output terminal 114, a change in the electrical resistance value of the ink detection sections 38 and 39, or a change in the electrical resistance value of the ink residual quantity detection section 36. The controller 117 reads out a processing program from the ROM 116 and stores it in the RAM 115 and performs respective processes based on the program.

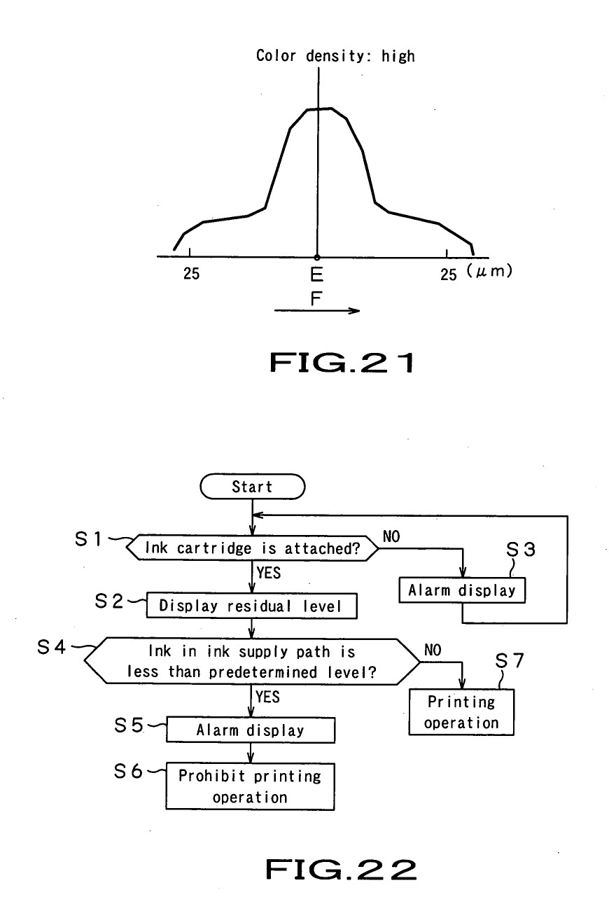

The controller 117 reads out a processing program for performing a discharge control from the ROM 116 and stores it in the RAM 115. Based on the program, the controller 117 switches ON/OFF of the switching elements 121a, 121b, 121c of the discharge control section 112 to periodically or randomly change the discharge direction of the ink droplet i. Under the control of the controller 117, the discharge control section 112 periodically or randomly change the discharge direction of the ink droplet i such that the ink droplet i is put on the recording paper P in a stopped state with density distribution approximated to the standard deviation distribution as shown in FIG. 21. More specifically, the controller 117 controls the switching elements 121b, 121c of the discharge control section 112 to periodically or randomly change the discharge direction of the ink droplet i such that the color density becomes highest (in other words, deepest in color) at the position E which is located on the recording paper P in the substantially perpendicular direction of the nozzle 104a of the head chip 41 and the color becomes deeper in the range of about a total of 10 μm, both backward and forward direction with respect to the position E, that is, in the direction (indicated by the arrow F in FIG. 21) substantially perpendicular to the feeding direction of the recording paper P. In the concrete, the controller 117 controls the switching element 121b formed in the sub-operation control element formation area 222 and controls the heating amount of the heating element 102a through the control wiring patterns 236, 236, 236 shown in FIG. 17.

Although the processing program is stored in the ROM 116 in the control circuit 110 having the above configuration, the medium that stores the program is not limited to the ROM. For example, it is possible to use, as the medium for storing the program, an optical disk, a magnetic disk, a magnetic optical disk, an IC card. In this case, the control circuit 110 is connected to a drive that drives the above recording media directly or through the information processor 118 and reads out the program from the recording media.

The entire operation of the printer apparatus 1 having the configuration described above will next be described with reference to a flowchart of FIG. 22. The present operation is performed by a not-shown CPU (Central Processing Unit) in the controller 117 according to the processing program stored in the storage means such as the ROM 116.

Firstly, a user selects text data, printing data or the like to be printed and starts printing operation through the information processor 118. Then the information processor 118 generates printing data based on the selected data and outputs the generated printing data to the input/output terminal 114 of the printer apparatus 1.

Next, the controller 117 determines, in step S1, whether predetermined ink cartridges 11y, 11m, 11c and 11k are attached to the attachment portions 32y, 32m, 32c, and 32k, respectively based on the engagement state between the projection 23 of the engagement projection portion 21 and engagement concave portion 24. When determining that all the ink cartridges 11 are adequately attached to the attachment portions 32, the controller 117 advances to step S2. On the other hand, when determining that at least one ink cartridge 11 is not adequately attached to the attachment potion 32, the controller 117 advances to step S3. In step S3, the controller 117 allows the alarm section 113 to perform an alarm display to notify the user of the ink cartridge 11 of the color that is not adequately attached.

In step S2, the controller 117 detects a change in the electrical resistance value of the ink quantity residual detection section 36. When detecting a change in the electrical resistance value, the controller 117 changes the display of the ink residual quantity according to the change in the electrical resistance value. Here, three ink residual quantity detection sections 36 are provided in the height direction of the ink cartridge 11, so that the controller can allow the alarm section 13 to perform the display of the residual quantity at three levels.

In step S4, the controller 117 determines whether the ink 4 in the connection portion 37 is less than a predetermined level, that is, whether it is a no-ink state or not. When determining that it is a no-ink state, the controller 117 allows the alarm section 113 to display the corresponding information, that is, perform an alarm display in step S5, and prohibits printing operation in step S6.

When detecting that the ink 4 in the connection portion 37 is not less than a predetermined level, that is, the connection portion 37 is still filled with the ink 4, the controller 117 permits the printing operation in step S7.

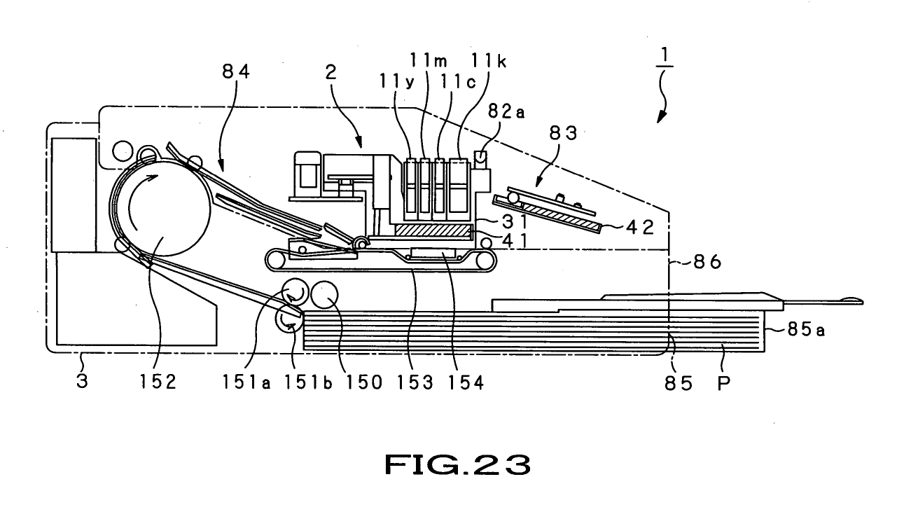

More specifically, as shown in FIG. 23, the controller 117 drives a drive motor that constitutes the head cap opening/closing mechanism 83 to move the head cap 42 to the tray 85a side, relative to the head cartridge 2 and allows the nozzle 104a of the discharge head 41 to be exposed. The controller 117 then drives a drive motor that constitutes the paper feed/eject mechanism 84 to continuously or intermittently feed the recording paper P. That is, the controller 117 draws out one recording paper P from the tray 85a using a paper feed roller 150 and allows a pair of separation rollers 151a and 151b rotating in reverse directions to each other to feed the drawn out recording paper P to a reverse roller 152. After the feeding direction of the recording paper P is reversed by the reverse roller 152, a pressing means 154 stops the recording paper P on a feeding belt 153 at a predetermined position, thereby positioning the landing position of the ink 4.

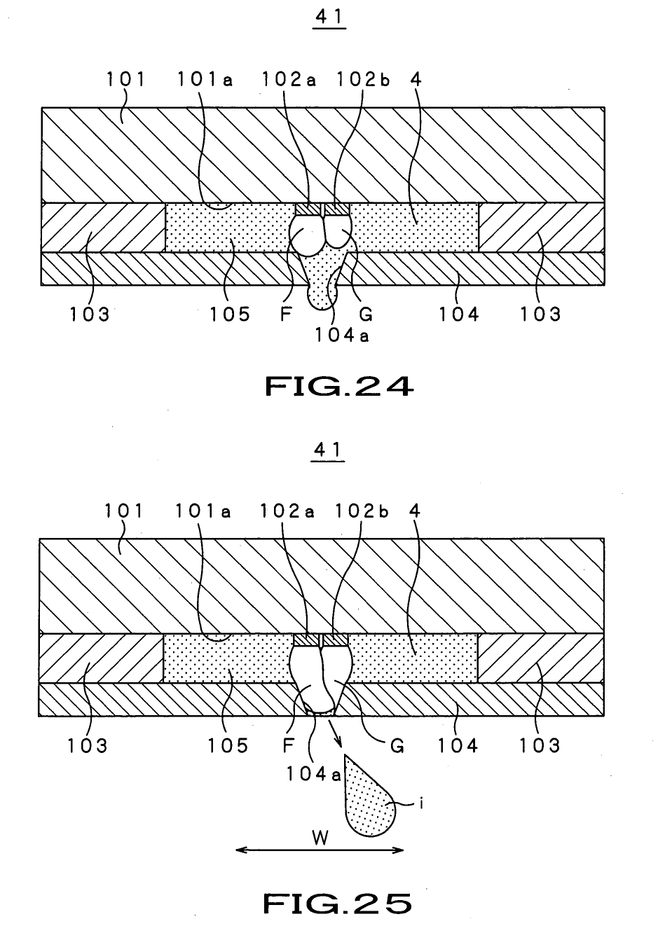

Simultaneously, the controller 117 controls the discharge control section 112 to discharge the ink droplet i onto the recording paper P from the discharge head 41. More specifically, as shown in FIG. 24, ink bubbles F and G are generated in the portion contacting the pair of heating elements 102a and 102b in the ink liquid chamber 105 to push aside the ink 4 by the volume corresponding to the expansion of ink bubbles F and G. As a result, the ink droplets i are discharged from the nozzle 104a by the volume corresponding to the volume of the pushed-side ink 4 contacting the nozzle 104a, and put on the recording medium such as the recording paper P, with the result that texts, images, and the like are printed on the recording paper P according to the printing data.

At this time, the discharge head 41 determines the discharge direction of the ink droplet i from the nozzle 104a depending on the expansion level of the respective ink bubbles F and G. In the discharge head 41, the ink bubble having a faster expansion speed presses the ink 4 more strongly, so that ink droplet i is discharged while being pushed to the side of the bubble having a slower expansion speed with respect to the nozzle 104a. Of the ink bubbles F and G, the ink bubble contacting the heating element that is heated more rapidly is expanded more quickly. Under the ON/OFF control of the controller 117 for the switching elements 121b and 121c that constitute the sub-operation controller, the discharge head 41 discharges the ink droplets i while periodically or randomly changing the discharge direction of the ink 4 from the nozzle 104a in the direction substantially perpendicular to the feeding direction of the recording paper P. With the above configuration, it is possible to prevent occurrence of blank line and irregularity in image quality due to variation in the discharge direction of the ink droplets i from the nozzles 104a of the discharge head 41. As a result, a high-quality image can be obtained.

When the ink droplet i is discharged as described above, the ink 4 is immediately replenished, by the quantity corresponding to the discharged quantity of the ink droplets i, to the ink liquid chamber 105 from the ink flow path 106 and the ink liquid chamber 105 is restored to its original state as shown in FIG. 10. When the ink droplet i is discharged from the discharge head 41 in the state where the valve 65 that is closing the opening portion 64 of the ink chamber 62 by the biasing forces of the biasing member 62 and diaphragm 69, the diaphragm 69 is pushed up by an atmosphere pressure under a negative pressure of the ink 4 as shown in FIG. 9, to thereby push up the valve shaft 68 and valve 65 against a biasing force of the biasing member 66. At this time, the opening portion 64 between the ink inflow path 61 side of the ink chamber 62 and ink outflow path 63 side is released, with the result that the ink 4 is supplied from the ink inflow path 61 side to the ink outflow path 63 side and replenished to the ink flow path 106. Thereafter, the negative pressure of the ink 4 is decreased and the diaphragm 69 is restored to the original shape by its restoring force, with the result that the biasing force of the biasing member 66 moves down the valve shaft 68 and valve 65 in such a direction to close the ink chamber 62. As described above, when the negative pressure of the ink 4 is increased every time the ink droplets i are discharged, the above operation is performed in the valve mechanism 54.

In this manner, texts or images corresponding to printing data are sequentially printed on the recording paper P that is being fed by the paper feed/eject mechanism 84. When the printing operation is completed, the recording paper P is ejected from the paper eject port 86.

As described above, in the process of deflecting the discharge direction of the ink droplet i from the nozzle 104a, power of about 0.5 to 1 W is supplied to the heating elements 102a and 102b through the power supply wiring pattern 224 in order to drive the heating elements. In the present invention, as shown in FIG. 17, the power supply wiring pattern 224 is widely formed and thereby has a low resistance value, so that it is possible to suppress the heat generation, thereby preventing the elements and the like formed on the semiconductor substrate 101 from being adversely affected. Further, the connection pattern 225 connected to the mid point between the pair of heating elements 102a and 102b is connected to the sub-operation control element formation area 222 apart from the heating elements 102a and 102b by extending across the main operation control element formation area 221. As a result, the connection pattern 225 is made longer than in the case of FIG. 16, so that the heat from the heating elements 102a and 102b can sufficiently be radiated, thereby protecting the sub-operation control element formation area 222 from the heat.

Although the head cartridge 2 is detachably attached to the printer main body 3 and the ink cartridge 11 is detachably attached to the head cartridge 2 in the printer apparatus 1, the abovementioned discharge head 41 is applicable to the printer apparatus in which printer main body 3 and head cartridge 2 is integrally formed.

Although the present invention has been described with the printer apparatus that prints texts or images on the recording paper taken as an example, the present invention is widely applicable to other types of apparatus, provided that the apparatus discharges a drop of liquid. For example, the present invention is applicable to a discharge apparatus for DNA chip in liquid (Jpn. Pat. Appln. Laid-Open Publication No. 2002-34560) and a liquid discharge apparatus that discharges a liquid containing conductive particles for forming a minute wiring pattern of a print wiring substrate.

### INDUSTRIAL APPLICABILITY

As described above, according to the present invention, the power supply wiring for supplying power to the bubble generation means and the control wiring for controlling the main operation control means and sub-operation control means are provided in different conductive layers, so that it is possible to widely form the power supply wiring to reduce the resistance value thereof, resulting in a reduction in heat generation.

1. A liquid discharge head that gives energy to liquid in a liquid chamber using an energy generation element to discharge the liquid from a nozzle, comprising:
a plurality of energy generation elements provided in the liquid chamber;
an energy supply wiring that supplies energy to the energy generation elements;
main operation control means for driving the energy generation elements for the purpose of discharging the liquid from the nozzle;
sub-operation control means for controlling the discharge direction of the liquid to be discharged from the nozzle while changing the amount of the energy to be supplied to the energy generation elements or changing the timing of giving energy thereto; and
control means for controlling the main operation control means and sub-operation control means, wherein
the energy generation elements, main operation control means, sub-operation control means, and control means are provided on a single semiconductor substrate, and
the energy supply wiring and a control wiring that controls the main operation control means and sub-operation control means are provided in different conductive layers on the semiconductor substrate.

2. The liquid discharge head according to claim 1, wherein the main operation control means, sub-operation control means, and control means are arranged in sequence on the semiconductor substrate.

3. The liquid discharge head according to claim 1, wherein the main operation control means, sub-operation control means, and control means constitute at least one group and the groups are disposed adjacently to each other on the semiconductor substrate.

4. The liquid discharge head according to claim 1, wherein the energy supply wiring is a common wiring for supplying energy to a plurality of energy generation means.

5. A liquid discharge apparatus having a liquid discharge head that gives energy to liquid in a liquid chamber using an energy generation element to discharge the liquid from a nozzle, wherein
the liquid discharge head comprises:
a plurality of energy generation elements provided in the liquid chamber;
an energy supply wiring that supplies energy to the energy generation elements;
main operation control means for driving the energy generation elements for the purpose of discharging the liquid from the nozzle;
sub-operation control means for controlling the discharge direction of the liquid to be discharged from the nozzle while changing the amount of the energy to be supplied to the energy generation elements or changing the timing of giving energy thereto; and
control means for controlling the main operation control means and sub-operation control means,
the energy generation elements, main operation control means, sub-operation control means, and control means are provided on a single semiconductor substrate, and
the energy supply wiring and a control wiring that controls the main operation control means and sub-operation control means are provided in different conductive layers on the semiconductor substrate.

6. The liquid discharge apparatus according to claim 5, wherein the main operation control means, sub-operation control means, and control means are arranged in sequence on the semiconductor substrate.

7. The liquid discharge apparatus according to claim 5, wherein the main operation control means, sub-operation control means, and control means constitute at least one group and the groups are disposed adjacently to each other on the semiconductor substrate.

8. The liquid discharge apparatus according to claim 5, wherein the energy supply wiring is a common wiring for supplying energy to a plurality of energy generation means.
